(19) United States
(12) Patent Application Publication
(10) Pub. No.: US 2025/0339432 A1
(43) Pub. Date: Nov. 6, 2025

(54) QUINAZOLINONE COMPOUND AS BRAF INHIBITOR FOR THE TREATMENT OF ADVANCED SOLID CANCER OR METASTASES

(71) Applicant: Hoffmann-La Roche Inc., Little Falls, NJ (US)

(72) Inventors: Alessandro BRIGO, Binningen (CH); Martin KORNACKER, Basel (CH); Nicole A. KRATOCHWIL, Basel (CH); Stefani PETER, Luzern (CH); Piergiorgio Francesco Tommaso PETTAZZONI, Basel (CH); Caroline RYNN, Wigton (GB); Gabriel SCHNETZLER, Basel (CH); Michael Johannes Georg HOFMANN, Basel (CH)

(73) Assignee: Hoffmann-La Roche Inc., Little Falls, NJ (US)

(21) Appl. No.: 19/210,927

(22) Filed: May 16, 2025

Related U.S. Application Data
(63) Continuation of application No. PCT/EP2023/082007, filed on Nov. 16, 2023.

(30) Foreign Application Priority Data
Nov. 18, 2022 (EP) 22208354.5

(51) Int. Cl. A61K 31/517; A61K 9/20; A61K 9/28; A61K 31/4523; A61P 35/00; A61P 35/04

(52) U.S. Cl. CPC A61K 31/517; A61K 9/2009; A61K 9/2013; A61K 9/2018; A61K 9/2027; A61K 9/2054; A61K 9/28; A61K 31/4523; A61P 35/00; A61P 35/04

(57) ABSTRACT

The present invention is directed to (3R)—N-[2-cyano-4-fluoro-3-(3-methyl-4-oxo-quinazolin-6-yl)oxy-phenyl]-3-fluoro-pyrrolidine-1-sulfonamide or a pharmaceutically acceptable salt thereof, for novel uses in the treatment of locally advanced solid tumours, in particular melanoma with brain metastases. The present invention also relates to pharmaceutical composition comprising (3R)—N-[2-cyano-4-fluoro-3-(3-methyl-4-oxo-quinazolin-6-yl)oxy-phenyl]-3-fluoro-pyrrolidine-1-sulfonamide or a pharmaceutically acceptable salt thereof.

The present invention provides a BRAF inhibitor of formula (I),

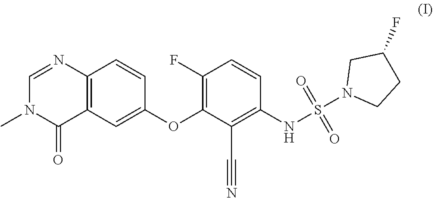 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of melanoma with brain metastases.

The invention further relates to novel methods and uses of the compound of formula (I) as defined above, as well as to pharmaceutical formulations comprising the compound of formula (I).

The chemical name of the compound of formula (I) is (3R)—N-[2-cyano-4-fluoro-3-(3-methyl-4-oxo-quinazolin-6-yl)oxy-phenyl]-3-fluoro-pyrrolidine-1-sulfonamide. Herein, the compound of formula (I) is also referred to as Compound Ia.

### BRIEF DESCRIPTION OF FIGURES

FIG. 1 Overview of phase Ia/b study design.

FIG. 2 Manufacturing process flow chart for a film-coated tablet comprising Compound Ia.

FIG. 3 Preliminary PK data regarding the food effect on PK of Compound Ia.

FIG. 4 Simulation based on a PopPK model to estimate Ctrough coverage of Compound Ia.

### BACKGROUND

Oncogenic mutations in the v-Raf murine sarcoma viral oncogene homolog B1 (BRAF) kinase gene have been observed in approximately 8% of all solid tumors. Such mutations result in constitutive activation of the BRAF kinase, which causes dysregulated downstream signaling via the MAPK/extracellular signal-regulated kinase (MEK) and the extracellular signal-regulated kinase (ERK), leading to excessive cell proliferation and survival. Three approved BRAF inhibitors (BRAFi) showed efficacy in indications such as BRAF V600E/K-positive melanoma. However, the low survival rates of patients with metastatic melanoma, including those whose tumors harbor the BRAF oncogene, highlights the need for improved drugs that target these mutations.

One of the most common and serious complications of cutaneous melanoma is the development of metastatic sites in the central nervous system (CNS). While the recently developed BRAFi significantly increase median progression-free survival (PFS) in patients with metastatic melanoma, the disease control is significantly shorter in patients with melanoma brain metastases, including in those with BRAF-mutant melanoma with brain metastases, compared with patients with extracranial disease only. Disease progression most often occurs in the CNS for brain metastatic patients under BRAFi therapy. Furthermore, patients with BRAF-mutated melanoma failing both checkpoint inhibitor (CPI) therapy and BRAF-targeted therapy (i.e., 3rd-line patients), have very few therapeutic options and represent a patient population with a high unmet medical need.

Compound Ia, which was previously disclosed in WO2021116055A1, is a potent RAF inhibitor targeting mutant BRAF V600E/K designed to avoid paradoxical MAPK induction in non-BRAF V600E/K-mutant cells and to present with high brain penetration to achieve efficacious drug exposures in the CNS. The present invention relates to new uses of the Compound Ia for the treatment of locally advanced solid tumours and/or metastatic cancer, in particular cutaneous melanoma with brain metastases, as well as suitable pharmaceutical compositions comprising the Compound Ia. Our preliminary data of the present phase Ia/Ib clinical trial suggests that Compound Ia has an excellent safety profile with less than 25% drug related Grade 3 adverse events, which is surprisingly far lower when compared to the currently approved BRAF inhibitors. In view of its excellent safety profile, the present compound has the potential to be dosed in a dose that provides for a much higher inhibitory concentration coverage and thus has the potential to provide significant benefit to the patients in terms of efficacy and/or safety.

The Compound Ia exhibits a low and pH dependent solubility and behaves like a weak acid across the physiological pH range that is purely soluble at low pH with increasing solubility at neutral and alkaline pH. Accordingly, one particular embodiment of the invention relates to a film-coated tablet comprising the Compound Ia, wherein the tablet composition further comprises a pH-modifier and thereby enhances the dissolution properties of Compound Ia. A certain embodiment of the present invention relates to an efficient and safe dosage regime of the Compound Ia, alone or in combination with cobimetinib, for the treatment of solid tumours and in particular melanoma with brain metastases.

### DETAILED DESCRIPTION

The term “inhibitor” denotes a compound which competes with, reduces or prevents the binding of a particular ligand to particular receptor, or which reduces or prevents the function of a particular protein. In particular, an inhibitor as used therein refers to compounds which target, decrease or inhibit activity of the respective target selected from BRAF and MEK, particular inhibitors have an IC50 value below 1 μM, below 500 nM, below 200 nM, below 100 nM, below 50 nM, below 25 nM, below 10 nM, below 5 nM, 2 nM or below 1 nM. In some embodiments of the invention the term “BRAF inhibitor” refers to compounds that decrease BRAF kinase activity at least about 10%, at least about 20%, at least about 30%, at least about 40%, at least about 50%, at least about 60%, at least about 70%, at least about 75%, at least about 80%, at least about 85%, at least about 90%, at least about 95% or at least about 99%. In some embodiments of the invention the term “MEK inhibitor” refers to compounds that decrease MEK kinase activity at least about 10%, at least about 20%, at least about 30%, at least about 40%, at least about 50%, at least about 60%, at least about 70%, at least about 75%, at least about 80%, at least about 85%, at least about 90%, at least about 95% or at least about 99%.

The term “IC50” refers to the concentration of a particular compound required to inhibit 50% of a specific measured activity. Similarly, IC80, IC90 and IC95, refer to the concentrations at which 90%, 90% and 95 of a specific measured activity are inhibited.

The term “BRAF mutant refers to those BRAF mutations that are involved in causing cancer, such as for instance BRAF V600E and V600K mutations.

The term “pharmaceutically acceptable salt” refers to those salts of the compound of formula (I) or of the MEK inhibitor which retain the biological effectiveness and properties of the free bases or free acids, which are not biologically or otherwise undesirable. These salts can for instance be formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of a compound of formula (I) are the hydrochloride salts, methanesulfonic acid salts and citric acid salts. Particular pharmaceutically acceptable salts of [3,4-difluoro-2-(2-fluoro-4-iodoanilino)phenyl]-[3-hydroxy-3-[(2S)-piperidin-2-yl]azetidin-1-yl]methanone or cobimetinib are the fumarate salts and succinate salts, in particular hemifumarate salts and hemisuccinate salts.

A certain embodiment of the invention relates to the compound of formula (I)

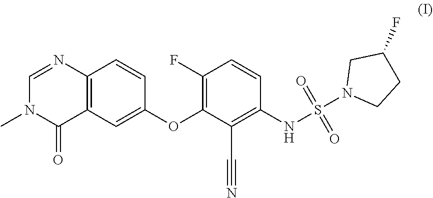 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, wherein the patient suffering from said cancer was previously under treatment with a different BRAF inhibitor.

A certain embodiment of the invention relates to the compound of formula (I)

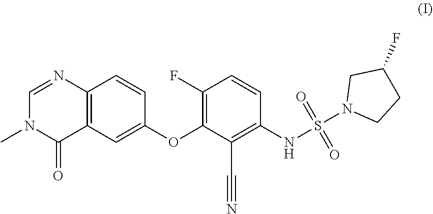 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, wherein the patient suffering from said cancer was previously not treated with a BRAF inhibitor, in particular a BRAF inhibitor selected from dabrafenib, vemurafenib and encorafenib.

A certain embodiment of the invention relates to the compound of formula (I)

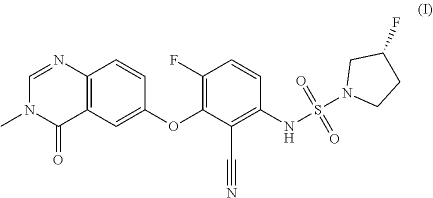 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of cancer, in particular melanoma with brain metastases, wherein the compound of formula (I) is combined with a MEK inhibitor.

Non-limiting examples of MEK inhibitors for the use according to the invention include cobimetinib, binimetinib, trametinib, selumetinib, pimasertib, refametinib, N-[2(R),3-dihydroxypropoxy]-3,4-difluoro-2-(2-fluoro-4-iodophenylamino)benzamide (PD-325901), 2-(2-chloro-4-iodophenylamino)-N-(cyclopropylmethoxy)-3,4-difluorobenzamide (Cl-1040) and 3-[2(R),3-dihydroxypropyl]-6-fluoro-5-(2-fluoro-4-iodophenylamino)-8-methylpyrido[2,3-d]pyrimidine-4,7(3H,8H)-dione (TAK-733). In one embodiment of the present invention the MEK inhibitor is cobimetinib. Cobimetinib is an orally available, potent and highly selective inhibitor of MEK1 and MEK2, central components of the RAS/RAF pathway. Cobimethib has the chemical name [3,4-difluoro-2-(2-fluoro-4-iodoanilino)phenyl]-[3-hydroxy-3-[(2S)-piperidin-2-yl]azetidin-1-yl]methanone and has the following structure:

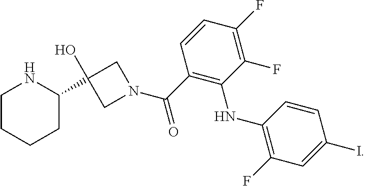 [SMILES: O=C(c1ccc(F)c(F)c1Nc1ccc(I)cc1F)N1CC(O)([C@@H]2CCCCN2)C1]

Cobimetinib may be prepared following the methods described in WO 2007/044515. Cobimetinib is commercially available and has the following CAS Registry Number: 934660-93-2.

In one embodiment of the present invention the MEK inhibitor is binimetinib. Binimetinib is an orally available, potent and highly selective inhibitor of MEK1 and MEK2, central components of the RAS/RAF pathway. Binimetinib has the chemical name 5-[(4-bromo-2-fluorophenyl)amino]-4-fluoro-N-(2-hydroxyethoxy)-1-methyl-1H-benzimidazole-6-carboxamide and has the following structure:

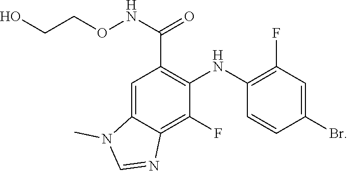 [SMILES: Cn1cnc2c(F)c(Nc3ccc(Br)cc3F)c(C(=O)NOCCO)cc21]

Binimetinib may be prepared following the methods described in WO 2003/077914. Binimetinib is commercially available and has the following CAS Registry Number: 606143-89-9.

In some embodiments, the checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (GP-675,206). In some embodiments, the PD-1 inhibitor is selected from pembrolizumab (Keytruda®), nivolumab (Opdivo®) and RN888. In some embodiments, the PD-L1 inhibitor is selected from atezolizumab (Tecentriq®), avelumab (Bavencio®) and durvalumab (Imfinzi™). In some embodiments, the PD-L1 inhibitor is atezolizumab (Tecentriq®).

### Abbreviations

BID=bis in die (latin), twice a day; PCR=polymerase chain reaction; PD=pharmacodynamics; PK=pharmacokinetics; PO=per os (latin), orally; QD=quaque die (latin), once daily; TID=ter in die (latin), three times a day.

### Test Agents:

The compound Ia can be synthesized according to the procedures described in WO2021116055A1 or according to methods known to those skilled in the art.

The following examples and figures are provided to illustrate the invention and have no limiting character.

### Biological Example—Phase IA/B Open-Label Study to Evaluate Safety, Pharmacokinetics, and Preliminary Clinical Activity of Compound Ia in Solid Tumours with BRAF-V600 Mutation

Part 1—Pilot PK: The dose-escalation has started with a Pilot PK Cohort in 2 participants to assess the PK of Compound Ia at a single dose of 25 mg with the aim to refine the starting dose for the dose escalation by using the actual exposure in humans and at least a 2-fold safety margin to the redefined human equivalent dose for the safety benchmark dosage of 200 mg/kg/day in rats. Once PK sampling was completed (maximal up to 72 hours after the first dose), participants will continue with daily Compound Ia treatment. Participants in the Pilot PK Cohort were enrolled and treated in a staggered manner (at least 3 days between the first and second participant). The preliminary analysis of the available PK data from the first 2 participants indicated that drug exposure at 25 mg is lower than anticipated giving rise to a 32- or 174-fold safety margin based on preliminary unbound Cₘₐₓ or AUC_{0-∞}, respectively, to the safety benchmark dose of 200 mg/kg/day (toxic dose in 10% of animals) in rats. No dose-limiting toxicities (DLTs) occurred at 25 mg Compound Ia in this Cohort. The dose for the first dose-escalation cohort was therefore newly defined by applying a 4- or 22-fold safety margin based on preliminary unbound Cₘₐₓ or AUC_{0-∞}, respectively, from the first two patients at 25 mg. The dose for first dose-escalation cohort will be 200 mg QD administration of Compound Ia. Following the completion of the Pilot PK Cohort DLT period on 25 mg QD dose, the two participants were included in the dose-escalation part of the study with Compound Ia, on 200 mg QD dose. They were considered DLT evaluable for this dose, with the 14 day DLT period starting at the point where they escalated the Compound Ia dose from 25 mg to 200 mg. No DLTs occurred during the 14 days after the Compound Ia was increased from 25 mg to 200 mg.

Part 1 (Dose-Escalation): Escalating doses of Compound Ia alone (Part 1a, monotherapy) and in combination with cobimetinib (Part 1b) will be assessed in participants with BRAF-V600 mutation-positive solid tumors (e.g., melanoma, non-small cell lung cancer (NSCLC), thyroid cancer, colorectal cancer (CRC) [with or without brain metastases]) to determine the maximum tolerated dose (MTD) and/or recommended Phase 2 dose (RP2D). The starting dose for the dose escalation was refined by using the actual exposure at 25 mg from the first two patients from 2 participants in the Pilot PK cohort. The Dose-Escalation Part will commence with once daily (QD) administration of Compound Ia at 200 mg dose. The dosing regimen will then change to a twice-daily (BID) or three-times-daily (TID) administration, depending on new emerging PK data.

Part 1a—Compound Ia Monotherapy Dose-Escalation: Dose-escalation will be carried out according to a mCRM-EWOC design. The number of patients with leptomeningeal involvement of their tumours is capped at 33% in each cohort. To avoid exposing participants to undue risk of toxicity, the maximum allowable dose increment recommended by the mCRM EWOC (modified continual reassessment method escalation with overdose control) design will be 100% (i.e., a 2-fold increase). The maximum dose that will be explored is 4'000 mg/day of Compound Ia. If any dose level within the QD or BID regimen demonstrates unacceptable toxicity or unfavorable PK characteristics, additional cohorts of participants may be evaluated in a TID regimen. The mCRM-EWOC will be adapted to the new dosing regimen.

Part 1b—Compound Ia in Combination with Cobimetinib: Dose-Escalation Part 1b will run as a concurrent arm, where Compound Ia will be administered in combination with a standard dose of cobimetinib (60 mg QD for 21 consecutive days, followed by a 7-day break). The starting dose for Compound Ia in combination with cobimetinib will be at least one dose level below the latest dose cleared and deemed as tolerable based on review of all Part 1a data. Escalation of Compound Ia in combination with cobimetinib will be guided by the mCRM EWOC model until MTD and/or RP2D is reached. The maximum dose that will be explored is 4'000 mg/day of Compound Ia. If any dose level within the QD or BID regimen of Compound Ia and cobimetinib demonstrates unacceptable toxicity or unfavorable PK characteristics, an additional cohorts of participants may be evaluated in a TID regimen. The mCRM-EWOC will be adapted to the new dosing regimen. On the basis of emerging PK and safety data, other cobimetinib doses may be explored as well. As for the monotherapy cohorts, for the combination cohorts with cobimetinib as well, the number of patients with leptomeningeal involvement of their tumors is capped at 33% at the cohort level.

Part 1—Food Cohort: The food effect (FE) on the PK of Compound Ia will be evaluated in Part 1a (Compound Ia monotherapy) or in Part 1b (Compound Ia in combination with cobimetinib) at the MTD and/or RP2D, or at dose levels close to the RP2D and/or MTD of Compound Ia with or without cobimetinib. For exploring the effect of food intake, participants will receive Compound Ia after a high-fat meal (Group 1) or following a 10-hour fast (Group 2) using a parallel design. For this pilot FE evaluation, a minimum of 6 evaluable participants will be enrolled. Once PK sampling is completed (maximal up to 72 hours after the first dose) to investigate the FE, participants will continue with either daily, BID or TID Compound Ia treatment according to the selected dosing schedule.

Part 2 (Dose Expansion): Following the determination of the MTD and/or RP2D and the potential of a FE, study treatment will commence to assess the safety and preliminary clinical activity in the following four cohorts.

| Participant population in Part 2 (a) | Treatment | Cohort | N |
|---|---|---|---|
| Participants with cutaneous melanoma | Compound Ia | Expansion | 40 |
| with asymptomatic brain metastases | alone | Cohort 1 | |
| who have not received any BRAFi | | | |
| (BRAFi naive participants) | | | |
| Participants with cutaneous melanoma | Compound Ia | Expansion | 40 |
| with asymptomatic brain metastases | alone | Cohort 2 | |
| who have received prior treatment | | | |
| with a BRAFi | | | |
| Participants with cutaneous melanoma | Compound Ia | Expansion | 40 |
| with asymptomatic brain metastases | in | Cohort 3 | |
| who have not received any BRAFi | combination | | |
| (BRAFi naive participants) | with | | |
| | cobimetinib | | |
| Participants with cutaneous melanoma | Compound Ia | Expansion | 40 |
| with asymptomatic brain metastases | in | Cohort 4 | |
| who have received prior treatment | combination | | |
| with a BRAFi | with | | |
| | cobimetinib | | |

(a) Prior systemic anti-cancer therapy with anti-PD-1/PD-L1 based immunotherapy is allowed.

The investigational medicinal products are Compound Ia and cobimetinib. All participants will receive Compound Ia (25 or 200 mg/tablet) PO QD or BID on every day of each 28-day cycle. Participants will receive 60 mg (3×20 mg tablets) cobimetinib PO QD on Days 1 to 21 of each 28-day cycle, i.e., for 21 consecutive days, followed by a 7-day break.

On days where participants are required to come into the clinic, a single dose of Compound Ia and cobimetinib (if applicable) will be administered to participants in the clinic setting. On all other study days outside the above scheduled clinic visits, participants will self-administer Compound Ia and cobimetinib (if applicable) at home. For Compound Ia and cobimetinib doses to be self-administered at home, a sufficient number of tablets should be dispensed to the participant to last until the next clinic visit or through one cycle, at the Investigator's discretion. Participants will self-administer oral study treatments as follows: Participants should take Compound Ia and cobimetinib (if applicable) at approximately the same time each day, unless otherwise instructed. Participants will be instructed as to the number and strength of tablets to take, according to their assigned study cohort, dose level, and schedule. Participants will be asked to record the time and date that they take each dose in a medication diary. Participants are to return all unused tablets at each study visit to assess compliance.

Compound Ia Administration: All participants will receive Compound Ia (25 or 200 mg/tablet) orally (PO) QD with a glass of water on every day of each 28-day cycle. If a dose of Compound Ia is missed (i.e., not taken within 4 hours after the scheduled dosing time), the participant should resume dosing with the next scheduled dose. Missed or vomited doses will not be made up. On all clinic visit days that require a pre-dose blood draw for Compound Ia PK sampling and/or laboratory assessments, participants will be instructed to take their morning oral dose of study drug in the clinic after completion of the pretreatment assessments.

Cobimetinib Administration: Participants will receive 60 mg (3×20 mg tablets) PO QD on Days 1 to 21 of each 28-day cycle, i.e., for 21 consecutive days, followed by a 7-day break. If there are concerns about tolerability, a lower dose or an alternative dosing schedule of cobimetinib may be explored. Cobimetinib should be taken at approximately the same time each day with the morning Compound Ia dose and no later than 4 hours after the scheduled time. Cobimetinib should be swallowed whole with a glass of water and should not be chewed, cut, or crushed. If a dose of cobimetinib is missed (i.e., not taken within 4 hours after the scheduled dosing time), the participant should resume dosing with the next scheduled dose. Missed or vomited doses will not be made up. On all clinic visit days that require a pre-dose blood draw for cobimetinib PK (pharmacokinetics) sampling and/or laboratory assessments, participants will be instructed to take their morning oral dose of study drug in the clinic after completion of the pretreatment assessments.

Meals and Dietary Restrictions: Generally, for participants receiving Compound Ia, no dietary restrictions are foreseen. A preliminary assessment of the FE (food effect) on the PK of Compound Ia will be done in Part 1 to inform about potential dietary requirements for the dose expansion (Part 2) in case a FE is observed. For participants receiving cobimetinib, the use of grapefruit juice, a potent CYP3A4 enzyme inhibitor, is prohibited during the study and for 30 days after the last dose of study treatment. For Participants in Food Cohort in the Food Cohort of Part 1, the effect of food on the systemic exposure of Compound Ia will be explored close to the MTD and/or at RP2D or relevant dose levels for a minimum of 6 participants. The participants will receive Compound Ia either in fed or fasted condition at C1D1. Following an overnight fast of at least 10 hours, Compound Ia will be administered as a single dose either after a high fat meal (i.e., 800-1000 calories, 50% fat; meal should be eaten in 30 minutes or less) or while fasted at Cycle 1 Day 1. In both cases, Compound Ia should be taken with 240 mL of water. Additional water is permitted ad libitum except for the period 1 hour before to 1 hour after drug administration. In both cases, food should not be consumed for at least 4 hours after the dose. The food intake during the high-fat meal plus calories and fat content will be recorded.

Alcohol: Participants must abstain from alcohol for 24 hours before the start of dosing until after collection of the final PK and/or PD sample on the respective study day. Participants will be queried on a regular basis about their alcohol consumption and appropriate comments concerning this intake will be recorded.

### Participant Population

Two participant populations will be enrolled in this study. Part 1: participants with BRAF-V600 mutation-positive metastatic or locally advanced solid tumors. Part 2: participants with BRAF-V600 mutation-positive cutaneous melanoma with CNS metastases.

### Key Inclusion Criteria:

Male and female participants aged 18 years with life expectancy of >3 months who signed written informed consent.
Eastern Cooperative Oncology Group (ECOG) Performance Status 2.
Documented BRAF-V600 mutation status of tumor tissue preferentially using an FDA-approved or CE-IVD genetic test.
Confirmation of availability of archival tumor tissue for submission to the sponsor/central laboratory.
Adequate bone marrow and end-organ function and coagulation parameters.
Willingness to abide by contraceptive measures for the duration of the study.
Applicable for Part 1 only: Histologically confirmed advanced/metastatic solid tumor with measurable systemic disease per Response Evaluation Criteria in Solid Tumors (RECIST) v1.1 (extracranial disease) or modified response assessment in neuro-oncology for brain metastases (mRECIST-BM; intracranial disease).
Applicable for Part 2 only: Histologically confirmed cutaneous melanoma with radiologically confirmed, asymptomatic brain metastases measurable per mRECIST-BM. Stable or improved CNS disease symptoms for at least 14 days prior to start of study treatment.

### Key Exclusion Criteria:

For part 2 only: History of or current leptomeningeal metastases.
Any metastasis requiring immediate local intervention.
Uncontrolled tumor-related pain.
Ascites, pleural effusion, or pericardial effusion requiring medical intervention (including use of diuretics) within 6 months prior to study entry.
Active malignancy (other than the one under investigation) or a prior malignancy within the past two years prior to enrollment with some exceptions.
Active uveitis, or any history of serous retinopathy or retinal vein occlusion.
Current or past history of CNS disease unrelated to the malignancy under investigation, such as stroke, epilepsy, CNS vasculitis, or neurodegenerative disease.
Active autoimmune disease, or quiescent autoimmune disease with exacerbations/flares within 1 year prior to enrollment.
Significant cardiovascular disease within the 6 months prior to study treatment administration.
Systemic anti-cancer therapy or small-molecular therapeutic(s), including but not limited to chemotherapy, investigational drugs, hormonal therapy and radiotherapy, and antibody-based agents all within 2 weeks or at least 5 half-lives, whichever is shorter, prior to start of study treatment.
Treatment with stereotactic radiosurgery or craniotomy within 1 week prior to study treatment or treatment with whole brain radiotherapy within 3 months prior to study treatment. Participants with local therapy should have complete recovery with no neurological sequelae.
Radiation therapy to visceral metastases within 1 week prior to study treatment. Palliative radiotherapy is allowed.
Major surgical procedure other than for diagnosis within 2 weeks prior to initiation of study treatment, or anticipation of need for a major surgical procedure during the course of the study.
Spinal cord compression not definitively treated with surgery and/or radiation or previously diagnosed and treated spinal cord compression without evidence that disease has been clinically stable for 2 weeks prior to screening.
Contraindication to cobimetinib or known hypersensitivity to any formulation component of cobimetinib (if applicable).
Participants with known hypersensitivity to BRAFi and/or MEK inhibitors (MEKi).
Strong CYP3A inducers (including St. John's wort and hyperforin) are prohibited during study treatment and for 2 weeks after the last dose of cobimetinib or Compound Ia (whichever is later).
Concomitant treatment with anti-convulsants other than gabapentin, vigabatrin and levetiracetam are prohibited (e.g., carbamazepine, phenytoin, phenobarbital due to strong CYP3A induction) during study treatment and for 2 weeks after the last dose of cobimetinib or Compound Ia (whichever is later).
For combination treatment with cobimetinib, moderate and strong CYP3A inducers and inhibitors are prohibited during study treatment and for 2 weeks after the last dose of cobimetinib or Compound Ia (whichever is later).
Refractory nausea and vomiting, malabsorption, external biliary shunt, or significant small bowel resection that would preclude adequate study treatment absorption.
Uncontrolled diabetes or symptomatic hyperglycemia.
Any Grade 3 hemorrhage or bleeding event within 28 days of study treatment initiation.
History of human immunodeficiency virus (HIV) positivity.
Hepatitis B virus (HBV) infection (chronic or acute), defined as having a positive hepatitis B surface antigen (HBsAg) test at screening or a positive quantitative HBV DNA test.
Hepatitis C virus (HCV) infection (chronic or acute) defined as having a positive HCV antibody test and a positive HCV RNA test at screening.
Known active or uncontrolled bacterial, viral, fungal, mycobacterial (including but not limited to tuberculosis and atypical mycobacterial disease), parasitic or other infection (excluding fungal infections of nail beds), or any major episode of infection.

### Study Treatment Dosage, Administration, and Compliance:

For detailed instructions on drug preparation, storage, and administration, refer to the pharmacy manual.

Participants will need to visit the clinic every fortnight for the first 3 cycles and monthly thereafter.

On days where participants are required to come into the clinic:

For QD Compound Ia dosing: a single dose of Compound Ia and the single dose of cobimetinib (if applicable) will be administered in the clinic setting.

For BID Compound Ia dosing: The first dose of Compound Ia and the single dose of cobimetinib (if applicable) will be administered to participants in the clinic setting. The second dose of Compound Ia for that day will be self-administered at home 10-12 hours after the first dose.

For TID Compound Ia dosing:
The first dose of Compound Ia and the single dose of cobimetinib (if applicable) will be administered to participants in the clinic setting in the morning.
The second and the third doses of Compound Ia for that day will be self-administered at home 8-10 hours after the first dose and 6-8 hours after the second dose respectively. Important: the second administration has to happen after the 8 hour PK blood sampling. In case the 8-hrs blood sampling is not required any more (depending on emerging data and only after sponsor's communication), the second medication intake can happen 6-8 hours after the first dose.

On all other study days outside the above scheduled clinic visits:

Participants will self-administer oral Compound Ia and cobimetinib (if applicable) at home. A sufficient number of tablets will be dispensed to the participant to last until the next clinic visit.

Participants should take Compound Ia and cobimetinib (if applicable) at approximately the same time each day:

For QD Compound Ia dosing: in the morning, Compound Ia and cobimetinib (if applicable).

For BID Compound Ia dosing: in the morning, Compound Ia and cobimetinib (if applicable); Compound Ia only in addition in the evening. Compound Ia should be taken 10-12 hours apart.

For TID Compound Ia dosing: in the morning, Compound Ia and cobimetinib (if applicable). Compound Ia only in addition, in the afternoon and evening. Compound Ia should be taken 6-8 hours apart.

Participants will be instructed as to the number and strength of tablets to take, according to their assigned study cohort, dose level and schedule. Participants will be asked to record the time and date that they take each dose in a medication diary and this diary should be brought to the hospital at every study visit. Participants are to return all unused tablets at each study visit to assess compliance.

### Compound Ia Administration:

All participants will receive Compound Ia (25 or 200 mg/tablet) orally (PO) QD, BID or TID with a glass of water on every day of each 28-day cycle. If a dose of Compound Ia is missed (i.e., not taken after the scheduled dosing time within 10 hours for QD; within 4 hours for BID; no window for TID), the participant should resume dosing with the next scheduled dose. Missed or vomited doses will not be made up.

On all clinic visit days that require a pre-dose blood draw for Compound Ia PK sampling (see Section 1.3) and/or laboratory assessments, participants will be instructed to take their morning oral dose of study drug in the clinic after completion of the pretreatment assessments.

In the case of BID and TID dose, all pre-dose and post-dose blood draws and assessments described in the protocol and in the SoA refer to the morning dose (first dose of the day).

### Cobimetinib Administration:

Participants will receive 60 mg (3×20 mg tablets) PO QD on Days 1 to 21 of each 28 day cycle, i.e., for 21 consecutive days, followed by a 7 day break. If there are concerns about tolerability, a lower dose or an alternative dosing schedule of cobimetinib may be explored.

Cobimetinib should be taken at approximately the same time each day with the morning Compound Ia dose and no later than 4 hours after the scheduled time. Cobimetinib should be swallowed whole with a glass of water and should not be chewed, cut, or crushed. If a dose of cobimetinib is missed (i.e., not taken within 4 hours after the scheduled dosing time), the participant should resume dosing with the next scheduled dose. Missed or vomited doses will not be made up.

On all clinic visit days that require a pre-dose blood draw for cobimetinib PK sampling and/or laboratory assessments, participants will be instructed to take their morning oral dose of study drug in the clinic after completion of the pretreatment assessments.

### Number of Participants:

The exact number of participants will depend on the occurrence of DLTs and the number of dose-levels required to determine the MTD and/or the RP2D. The study will enroll a maximum of 120 participants in the dose-escalation stage (60 participants each for Part 1a and Part 1b) and up to approximately 40 participants in up to 4 expansions in Part 2. For FE, an additional cohort of 6-12 participants will be enrolled. The overall number of participants will be, at most, 292.

### Concomitant Medications:

Any medication or vaccine used by a participant within 30 days of screening until safety follow-up visit must be recorded along with a reason for use, dates of administration and dosage information. As a general rule, no new concomitant medication will be permitted, with the exception of medications to treat AEs, unless the rationale for exception is discussed and clearly documented between the Investigator and the Sponsor. If deemed necessary for the treatment of brain metastases, systemic corticosteroids, i.e., doses of ≤8 mg/day of dexamethasone or equivalent, and anti-epileptic drugs (i.e. gabapentin, vigabatrin and levetiracetam) may be administered at the discretion of the treating physician.

### Justification for Dose:

Based on currently available non-clinical drug safety information and predicted human PK, the starting dose of Compound Ia was 25 mg administered QD, which was expected to be safe and pharmacologically active in participants. Due to the uncertainty in predicted human PK (estimated half-life between 7 and 70 hours), the human dose range yielding equivalent unbound exposure to the safety benchmark dose of 200 mg/kg/day in rat was predicted to be 46 to 610 mg, respectively. By applying a 2-fold safety margin, a human starting dose of 25 mg was selected. In a Pilot PK Cohort prior to the dose-escalation in Part 1a (monotherapy), the PK of Compound Ia was explored after a single, oral dose of 25 mg in 2 participants to address the PK uncertainty. The preliminary analysis of the available PK data from the first 2 participants at 25 mg indicated that the drug exposure in human is lower than anticipated due to a short half-life estimated to be between 2 and 4 hours. Based on the preliminary PK characteristics of Compound Ia at 25 mg from the first 2 participants in the Pilot PK Cohort, the dose for the first dose-escalation cohort was newly defined as 200 mg QD. This was determined by using the actual exposure of Compound Ia in human at 25 mg and applying a 4- or 22-fold safety margin based on unbound Cₘₐₓ or AUC_{0-∞} to the no-observed-adverse-effect level (NOAEL) safety benchmark dose of 200 mg/kg/day in rat.

### Food Effect Assessment:

Compound Ia is a biopharmaceutics classification system class 2 drug and therefore the PK of Compound Ia may be affected by meals. The FE pilot assessment during dose-escalation at the MTD and/or RP2D or relevant dose levels aims to explore whether food has a significant impact on the PK of Compound Ia and to inform the expansion cohorts in Part 2 and future clinical studies in regard to dietary requirements for oral administration of Compound Ia.

### Efficacy Assessments:

In general, tumor assessments must be performed independently of changes to the study treatment administration schedule (i.e., when treatment is withheld). If a tumour assessment has to be performed early or late, subsequent assessments should be conducted according to the original schedule based on the date of first study treatment administration. Tumor assessments are to continue according to schedule in participants who discontinue treatment for reasons other than disease progression (unless subsequent anti-cancer therapy is initiated). An objective response will be confirmed by two consecutive assessments at least four weeks apart. Measurable lesions that have been previously irradiated will not be considered target lesions unless increase in size of 20% has been observed following completion of radiation therapy.

### Tumor and Response Evaluations of Extracranial Lesions:

Tumor assessments will be performed at the pre-defined time-points. All known sites of disease must be documented at screening and re-assessed at each subsequent tumor evaluation. The on-treatment extracranial tumor assessments at C2D1 (Cycle 2, Day 1) and C3D1 (Cycle 3, Day 1) must be performed with a time window of ±3 days and during subsequent timepoints with a time window of ±7 days. Response assessment will be performed according to the RECIST v1.1 for participants. The extent of neoplastic disease will be determined by reproducible radiographic techniques, preferably CT or MRI scan. Ultrasound and X-rays are not acceptable for monitoring target lesions. CT (or MRI) scans should include chest, abdomen, and pelvic scans, with additional examinations performed if clinically indicated (e.g., bone scans and CT scans of the neck). All known and suspected areas of neoplastic disease will be evaluated as deemed appropriate by the Investigator. For each participant, the same method of assessment and the same technique must be used consistently throughout the entire study. Use of spiral CT or MRI is required for screening lesions <20 mm and must be documented in medical records and used consistently throughout the study. The use of oral and IV contrast should, as long as it is clinically possible, be kept consistent. MRIs of the abdomen, and pelvis with non-contrast CT scan of the chest may be used in participants for whom CT scans with contrast are contraindicated (i.e., participants with contrast allergy or impaired renal clearance). If a CT scan for tumor assessment is performed using a PET/CT scanner, the CT should be of identical diagnostic quality as a full-contrast diagnostic CT scan. Assessments should be performed by the same evaluator if possible to ensure internal consistency across visits. At the Investigator's discretion, other methods of assessment of measurable disease as per RECIST v1.1 may be used. In case of clinically measurable superficial (such as skin) lesions, repeated photographs should be used to document tumor response. These photos must include a ruler for documentation purposes. Participants with known or suspected bone metastases should undergo radionuclide bone scanning. At the Investigator's discretion, CT scans may be repeated at any time if progressive disease is suspected.

### Tumor and Response Evaluations of Intra-Cranial Lesions:

Brain imaging will be performed at pre-defined time points. All known sites of disease must be documented at screening and re-assessed at each subsequent tumor evaluation. The on-treatment intra-cranial tumor assessments at C2D1 and C3D1 must be performed with a time window of ±3 days and during subsequent timepoints with a time window of ±7 days. Response assessment of brain metastasis will be performed according to the mRECIST-BM criteria with a modified measurability definition for intra-cranial lesions (≥0.5 cm by MRI) and allowing up to five intra-cranial target lesions (in addition to any extracranial target lesions). Brain imaging must be performed using MRI, with the following image acquisition requirements.

Minimum sequences required:
Pre-contrast T1, T2/FLAIR.
Post-contrast T1, with two orthogonal planes (or a volume acquisition) recommended.
The recommended slice thickness is ≤5 mm with no gap.

If extracranial progressive disease is detected, a brain MRI scan must also be completed within a week of extracranial PD determination. In addition to tumor and response evaluation for both extracranial and intra-cranial lesions, further analysis of the CT and MRI scans might be performed to further elucidate the drug effect.

### [18F] FDG-PET Imaging:

[18F]-FDG-PET will be performed at pre-defined time-points to determine drug effect on the tumor. The imaging acquisition guidelines should be followed as described in the Imaging Manual. The participant should fast for 4 to 6 hours prior to the [18F]-FDG-PET investigation. The participant will be resting in the waiting room after the tracer administration to be ready for the scanning starting 60 minutes±10 minutes after administration. The participant will be examined with a sufficient number of bed positions to ensure coverage over the area from the lower part of the brain (inclusive) to mid-thigh. It is also essential that the participant will be examined under the same conditions at screening and follow-up scans, specifically with respect to uptake time and scanning time. It is recommended that the same model of scanner is used or at least that the same spatial resolution is ensured. [18F]-FDG-PET/CT will be performed at screening. For on-study FDG-PET, this assessment must occur before Compound Ia administration at the specified time-points. The on-treatment assessments at C1D15 and C2D1 must be performed with a time window of −3 days. Participants with no evidence of FDG uptake on screening PET scan will not be required to undergo follow-up studies. For participants with suggestion of progressive disease on follow-up FDG PET assessments, a confirmatory CT/MRI assessment is strongly recommended. Response assessment using FDG-PET will be performed both at the sites and centrally by an independent reviewer. Transient unilateral axillary lymphadenopathy has been observed following mRNA-based SARS-CoV-2 vaccination and this may confound interpretation in oncology patients using CT, MRI, or PET/CT. While COVID-vaccination of study participants should not be delayed, consideration should be given to administering the vaccine at a location that avoids potentially confounding imaging findings, for example, in contralateral arm for localized cancers or in the thigh. Further, details regarding vaccination such as vaccination date, anatomic site, laterality and vaccine manufacturer (if available) should be collected at the time of imaging to facilitate interpretation.

### Safety Assessments:

Safety assessments will consist of monitoring and recording AEs, including serious AEs (SAEs) and non-SAEs of special interest (NSAESI); measurement of protocol-specified safety laboratory assessments; measurement of protocol-specified vital signs, ECGs; and other protocol-specified tests that are deemed critical to the safety evaluation of the study.

A complete physical examination will be conducted at screening and at the 28-day safety follow-up visit, and will include, at a minimum, assessments of the cardiovascular, respiratory, gastrointestinal, dermatological and musculoskeletal systems in addition to head, eyes, ears, nose, throat, neck and lymph nodes. The complete physical examination includes weight and height. A complete neurological examination at screening, which includes an evaluation of mental status, cranial nerves, strength, sensation, and coordination and gait should be performed and documented in the participant chart. Examination of other body systems may be performed in case of evocative symptoms at the Investigator's discretion. Neurological symptoms may include the following (but are not limited to): headache, nausea and/or vomiting, vertigo and/or dizziness, restlessness and/or irritability, fatigue or sleeplessness, hearing loss, muscle weakness, balance problems, speech problems, and others. Any abnormality identified at screening should be recorded. At other visits, targeted physical examinations should be performed. Targeted physical examinations should be limited to systems of primary relevance (i.e., cardiovascular, respiratory, neurologic, and any system that might be associated with tumor assessment [e.g., those systems associated with symptoms], or potential drug-related toxicity). Targeted neurologic examinations should include an evaluation of alertness and orientation to person, place, and time. This examination should also include a symptom-driven focused examination of other cranial nerves, strength, sensation, coordination, and mentation as clinically warranted. Of note, the targeted physical examination may encompass the targeted neurologic examination if relevant to the patient's health status. Where a targeted neurological examination is appropriate, this should be performed regardless of the patient's health status. Changes from baseline abnormalities should be recorded in participants' notes. New or worsened clinically significant abnormalities should be recorded as AEs. Investigators should pay special attention to clinical signs related to previous serious illnesses.

Vital Signs Vital signs will include measurements of systolic and diastolic blood pressure, respiratory rate, pulse rate, and body temperature while the participant is in a sitting or semi-supine position. Every effort should be made to ensure that vital signs are obtained from participants in a consistent manner/position. Blood pressure and pulse measurements should be measured after at least 5 minutes of sitting or lying supine in a quiet setting without distractions (e.g., television, cell phones; consistency should be maintained for an individual participant). Blood pressure and pulse measurements can be assessed with a completely automated device. Manual techniques will be used only if an automated device is not available. When possible, the same arm should be used for all blood pressure measurements. When measuring blood pressure, the participant's arm should be unconstrained by clothing or other material, and the participant should be comfortably seated, with the legs uncrossed, and the back and arm supported, such that the middle of the cuff on the upper arm is at the level of the right atrium (the midpoint of the sternum), the “ideal” cuff should have a bladder length that is 80% and a width that is at least 40% of arm circumference (a length-to-width ratio of 2:1).

### Electrocardiograms:

Triplicate 12-lead Electrocardiograms: Triplicate 12-lead ECG will be obtained using an ECG machine that automatically calculates the heart rate and measures pulse rate, QRS, QT, and QT corrected for heart rate (QTc) intervals. To minimize variability, it is important that participants be in a resting position for at least 10 min prior to each ECG evaluation. Body position should be consistently maintained for each ECG evaluation to prevent changes in heart rate. Environmental distractions (e.g., television, radio, conversation) should be avoided during the pre-ECG resting period and during ECG recording. ECGs should be performed 2 h after administration of study treatment and after the blood draws for PK/PD samples at the 2 h time point. In case of an absolute QTc of >500 ms and an increase from baseline QTc >60 ms, another triplicate ECG must be recorded within the next 30 min. It may be appropriate to repeat abnormal ECGs to rule out improper lead placement potentially contributing to the ECG abnormality. In case of QTc shortening (≤340 ms or >15% decrease from the baseline value), another triplicate ECG must be recorded within the next 30 min. ECG characteristics, including heart rate, QRS duration, pulse rate, and QT intervals, will be recorded. QTcF (Fridericia's correction) and RR interval will be automatically calculated and recorded. Changes in T-wave and U-wave morphology and overall ECG interpretation will be documented. T-wave information will be captured as normal or abnormal, U-wave information will be captured in two categories: absent/normal or abnormal. Additional PK samples should also be taken if clinically significant changes in ECHO or MUGA findings are identified.

Holter Electrocardiogram Monitoring Digital continuous recordings (Holter) for the extraction of 12-lead ECG traces will be performed at predefined timepoints. The optimal on-treatment timepoint of the Holter ECG may be reassessed with new emerging data and may be shifted or additional assessments may be added if considered necessary for clinical evaluation. Participants will wear a digital Holter monitoring device for continuous recordings of 12-lead ECG traces over a 24-hour period. Holter recordings will be sent to a central ECG analysis laboratory for ECG extraction and potential retrospective expert review with estimation of ECG intervals. At predefined timepoints during the 24-hour recording period, replicate ECG tracings will be extracted from the continuous recording during a 5-minute time window that coincides with the scheduled ECG time point of verified stability of the heart rate for 1 minute. At these specific timepoints, the participants should be at rest and in a supine position for ≥10 minutes before and remain in a supine position for ≥5 more minutes after the specified ECG extraction timepoints. Replicate estimates of uncorrected QT intervals and QTcF will be derived from the ECG traces at each scheduled timepoint, along with other intervals (PR, RR, and QRS) and information on T-wave and U-wave morphology as appropriate. While the participants are on Holter ECG monitoring, the absence of any environmental distractions (e.g., television, radio, conversation, or telephone calls) during the pre- and post-ECG rest period, and at predefined ECG timepoints must be emphasized. In particular, activities known to cause changes in heart rate should be avoided. Where predose and 2 hr post dose PK samples need to be taken on C1D8, the below sequence of events should be followed:

For Predose (up to 2 hr prior to the scheduled drug administration)
Take the Pre-dose PK sample and record the time of sampling
Fit the Holter device
Ensure the patient rests for 10 mins
Start the Holter
Ensure the patient rests for a further 5-10 mins
The central ECG laboratory will retrospectively extract an ECG trace from the Holter in the 5 min window after Holter is started

For 2 hr Post dose PK sample
Ensure the patient rests for 10 mins before and for 5 mins after the 2 hr time point
ECG
Then draw the actual PK blood sample
The central ECG laboratory will retrospectively extract an ECG trace from the Holter in the 5 min window around the scheduled timepoint (when the patient is resting).

The timings of assessments may be amended or the number of assessments increased during study conduct on the basis of emerging data to allow for optimal characterization of the effect profile.

### Evaluation of Left Ventricular Function:

Participants will undergo evaluation of left ventricular ejection fraction (LVEF), either by echocardiogram, or MUGA, and as clinically indicated for new or worsening symptoms. Any participant who develops clinical signs or symptoms suspicious of cardiac failure should undergo an LVEF re-assessment and further cardiovascular consultation, as needed. Additional PK samples should also be taken if clinically significant changes in ECHO or MUGA findings are identified. Evaluation of left ventricular function must be performed using the same method for each participant.

### Ophthalmologic Examinations:

For Compound Ia monotherapy the participants will be monitored for signs and symptoms of uveitis. Examinations will include visual acuity testing, intraocular pressure measurements by tonometry, slit-lamp ophthalmoscopy, specular microscopy and direct or indirect ophthalmoscopy. Ophthalmologic examinations must be performed by a medical professional trained in these assessments, or otherwise, the participant should be referred to a qualified ophthalmologist. For Compound Ia and Cobimetinib combination therapy the participants will be monitored for signs and symptoms of ocular toxicity including uveitis, serous retinopathy and retinal vein occlusion. Ophthalmologic examinations must be performed by a medical professional trained in these assessments, or otherwise referred to a qualified ophthalmologist. Examinations will include visual acuity testing, intraocular pressure measurements by tonometry, slit-lamp ophthalmoscopy, direct or indirect ophthalmoscopy as warranted, and spectral domain optical coherence tomography (OCT). If spectral domain OCT is not available, time-domain OCT may be performed instead. Fluorescein angiography may also be performed if warranted.

### Dermatologic Examinations:

Evaluations of the skin by a qualified medical specialist will be performed. An unscheduled dermatology examination may be performed during treatment for investigation of any new skin lesions, as clinically indicated. Participants with new, concerning skin lesions should be referred to a dermatologist for further assessment, diagnosis, and treatment.

### Clinical Safety Laboratory Assessments:

The clinical laboratory tests must be conducted in accordance with the local laboratories process. The Investigator must review the laboratory report, document this review, and record any clinically relevant changes occurring during the study. The laboratory reports must be filed with the source documents. Clinically significant abnormal laboratory findings are those which are not associated with the underlying disease, unless judged by the Investigator to be more severe than expected for the participant's condition.
In the event of unexplained abnormal clinically significant laboratory test values, the tests should be repeated immediately and followed up until they have returned to the normal range and/or an adequate explanation of the abnormality is found.
If laboratory values from non-protocol-specified laboratory assessments performed at the local laboratory require a change in participant management or are considered clinically significant by the Investigator (e.g., SAE or AE or dose modification), these results must be recorded in the eCRF.

Results of clinical laboratory testing will be recorded on the eCRF or be received as electronically produced laboratory reports submitted directly from the local or central laboratory. Additional blood or urine samples may be taken at the discretion of the Investigator if the results of any test fall outside the reference ranges or clinical symptoms necessitate additional testing to monitor participant safety. Where the clinical significance of abnormal laboratory results at screening is considered uncertain, screening laboratory tests may be repeated before the first dose of study treatment to confirm eligibility. Based on continuous analysis of the data in this study and other studies, any sample type not considered to be critical for safety may be stopped at any time if the data from the samples collected do not produce useful information.

### ECOG Performance Status:

ECOG performance status will be assessed at screening, at the discontinuation visit, and at pre-defined safety follow-up visits. It is recommended, where possible, that a participant's performance status will be assessed by the same person throughout the study. If ECOG performance status is performed within 72 hours prior to the scheduled dosing, it does not need to be repeated on the scheduled dosing day of that cycle. If performed on the scheduled dosing day, it must be done prior to the study treatment administration.

### Adverse Events and Serious Adverse Events:

Time Period and Frequency for Collecting Adverse Event and Serious Adverse Event Information: Investigators will seek information on AEs at each participant's contact. All AEs, whether reported by the participant or noted by study personnel, will be recorded.

Method of Detecting Adverse Events and Serious Adverse Events: Care will be taken not to introduce bias when detecting AEs and/or SAEs. Open-ended and non-leading verbal questioning of the participant is the preferred method to inquire about AE occurrence. A consistent methodology of non-directive questioning should be adopted for eliciting AE information at all participant evaluation timepoints.

### Pharmacokinetics:

Blood samples to evaluate concentrations of Compound Ia and cobimetinib (and its metabolite[s], if appropriate) will be collected. The date and time of each sample collection will be recorded. Compound Ia and cobimetinib levels will be analyzed by using validated LC-MS/MS assays. The PK samples will be taken as outlined in Table 13. Dedicated PK samples will be collected for the Pilot PK Cohort and the Food Cohort at pre-defined timepoints. During the course of the study, PK sampling timepoints may be modified on the basis of emerging data to ensure the PK of Compound Ia and cobimetinib can be adequately characterized. Additional PK samples will be taken at the following events:
At the time of treatment discontinuation.
If an AE Grade ≥3 leading to dose-reduction or delay of Compound Ia and/or cobimetinib administration is reported.
Clinically significant changes in ECGs/echocardiogram or MUGA scan.
In case of a DLT.
At the first occurrence of an objective response (i.e., PR or CR).
At the time of disease progression.

CSF samples to evaluate concentrations of Compound Ia (and its metabolite[s], if appropriate) will be collected from a dedicated set of participants. The date and time of each sample collection will be recorded in the eCRF. Compound Ia levels in CSF will be analyzed by using validated LC-MS/MS assays. The CSF samples will be taken as outlined in Table 13.
Metabolites may be measured by a specific validated LC-MS/MS assay, or other fit for purpose methods as appropriate.
Any leftover PK and CSF samples remaining after the specified analyses may also be used for additional validation experiments (e.g., metabolite identification).

If required, remaining PK and CSF samples may also be used for assay development/validation experiments. The PK and CSF blood samples will be destroyed within 2 years after the date of the clinical study report (CSR), unless the participant agrees otherwise.

### Pharmacodynamics and Biomarker Analyses:

Archival formalin-fixed paraffin-embedded (FFPE) tumor tissue will be collected from all participants in the study for NGS, using the F1CDx, a qualitative NGS FDA-approved tissue-based broad companion diagnostic (CDx). Analysis techniques may include, but are not limited to, BRAF-V600 mutation status, microsatellite instability (MSI) and tumor mutation burden (TMB) in tumor. Blood and CSF samples for PD will be collected as specified in Table 13. The date and time of each sample collection should be recorded in the eCRF. Details on processes for collection and shipment of these samples can be found in the Laboratory Flow Chart. These samples will be tested for DNA or blood and CSF-derived biomarkers relating to PD and mechanism of action of Compound Ia and cobimetinib. The samples include, but are not limited to, assessing BRAF-V600 mutation status, MSI, TMB and ctDNA monitoring in blood and CSF. Analysis techniques may include, but are not limited to NGS and PCR. These analyses will be performed using the F1-Tracker, which uses Natera's Signatera, a personalized multiplex PCR that leverages the power of F1CDx to define a set of tumor-specific variants that will subsequently be monitored using Signatera ctDNA monitoring assessment and the F1LCDx, a qualitative NGS FDA-approved test for liquid biopsy. PD assessments may be revised during the course of the trial depending on assay availability, emerging data, and results from the initial analysis. The samples may also be used for research purposes to identify biomarkers useful for predicting and monitoring response to Compound Ia and cobimetinib treatment, identifying biomarkers useful for predicting and monitoring Compound Ia and cobimetinib safety, assessing PD effects of Compound Ia and cobimetinib treatment, and investigating mechanism of therapy resistance. Additional biomarkers may be measured in the case that a strong scientific rationale develops. During the course of the study, sampling time points may be modified on the basis of emerging data to ensure the PD of Compound Ia can be adequately characterized (but without increasing overall blood collection volume). When a participant withdraws from the study, samples collected prior to the date of withdrawal may still be analyzed, unless the participant specifically requests that the samples be destroyed or local laws require destruction of the samples. However, if samples have been tested prior to withdrawal, results from those tests will remain as part of the overall research data. Data arising from all biosamples including samples for analyses of inherited DNA will be subject to the study confidentiality standards.

### Endogenous CYP3A Activity Markers Analyses:

Mandatory blood samples to measure concentrations of 4βHC, TC and/or 4αHC will be collected to assess the CYP3A activity during Compound Ia treatment. The date and time of each sample collection will be recorded in the eCRF. 4βHC, TC and/or 4αHC levels will be analyzed by using validated LC-MS/MS assays. The samples will be taken as outlined in Table 13. During the course of the study, sampling timepoints may be modified on the basis of emerging data to ensure the CYP3A activity during Compound Ia treatment can be adequately characterized. The blood samples will be destroyed after the date of final clinical study report or after approval by the study management team, or earlier depending on local regulations. Details on sampling procedures, sample storage, and shipment are given in the sample documentation.

### Genetic and Genomic Analyses:

Archival FFPE tumor tissue for DNA analysis will be collected before study treatment administration. The DNA may be used for, but not limited to, the detection of the genetic mutation of BRAF-V600 and exploratory research on genetic biomarkers by the F1CDx. Whole blood samples for DNA analysis will be taken, at baseline or before study treatment administration and in Part 2 at discontinuation. The DNA may be used for, but not limited to BRAF-V600 mutation status and exploratory research on genetic biomarkers by the F1LCDx. For the F1CDx and F1LCDx tests, samples and/or their derivatives may be sent to one or more laboratories for analysis of germline or somatic mutations via next-generation sequencing (NGS) or other genomic analysis methods, that form the basis of F1CDx and F1LCDx. Exploratory research on genetic biomarkers may include, but is not limited to, cancer-related genes, biomarkers associated with common molecular pathways, MSI status, and TMB. Whole blood samples for ctDNA analysis will be taken during the study treatment. Analysis of cell-free DNA (cfDNA) extracted from the whole blood samples may include, but is not limited to, mutations of genes associated with cancer progression, tumor molecules per mL, number of positive mutations defined by the F1-Tracker, that may affect the PK, PD, clinical activity, or safety of the study treatment. For this purpose, samples and/or their derivatives may be sent to one or more laboratories for analysis by multiplex PCR or other genomic analysis methods, which form the basis of the F1-Tracker. Genomics is increasingly informing researchers' understanding of disease pathobiology. Target DNA analysis provides a comprehensive characterization of the genome and, along with clinical data collected in this study, may increase the opportunity for developing new therapeutic approaches. Data will be analyzed in the context of this study but may also be explored in aggregate with data from other studies. The availability of a larger dataset will assist in identification of important pathways, guiding the development of new targeted agents. Given the complexity and exploratory nature of these analyses, data and analyses will not be shared with investigators or study participants unless required by law. Participants will not be identified by name or any other personally identifying information. Data arising from all biosamples including samples for analyses of inherited DNA will be subject to the confidentiality standards described in the sample documentation. Participants will not be identified by name or any other personally identifying information. Data arising from all biosamples, including samples for analyses of inherited DNA, will be subject to the confidentiality standards described in the sample documentation.

### Pharmacodynamics and Biomarker Samples:

Blood, CSF, and archival biopsies samples will be collected. The date and time of each sample collection should be recorded. In addition, any residual material from blood may be used for retrospective and longitudinal testing of bacterial or viral infection by serological methods. This testing may be performed for each participant. In addition to serving as an important safety measure, these analyses will inform as to any association of bacterial or viral infection and response to treatment. Any residual material from blood and tumor tissue samples may be also used for other exploratory analyses and research to find new biomarkers or to develop these laboratory tests for this study drug or similar drugs after the mentioned intended uses. Unless otherwise specified below, samples (including blood, slides, extracts, etc.) will be destroyed no later than 5 years after the final clinical study report, unless the participant provides specific consent for the remainder of the material to be stored for optional exploratory research.

### Blood Sampling:

The following blood samples for plasma isolation will be collected for PD and exploratory biomarker assessments:
Blood for BRAF V600 mutation assessment will be taken for DNA extraction and clinical genotyping (confirmation of BRAF V600 mutation) from every participant. The sample should be collected before start of treatment as specified in the SoA. If the sample is not collected on C1D1 pre-dose, it can be collected at any other scheduled visit at the earliest possible time.
Blood for ctDNA will be taken for DNA extraction and ctDNA assessment from every participant at screening, on treatment, and at the time of progressive disease as specified in the SoA.
Blood samples to measure concentrations of 4βHC, TC and/or 4αHC will be collected to assess the CYP3A activity during Compound Ia treatment.

Tissue Sampling Archival Tumor tissue should preferably come from the primary tumor or, if not available, from a prior metastasis. The archival specimen can be of any age and must contain adequate viable tumor tissue to establish BRAF V600 mutation status by a central laboratory. In addition, these samples will be used to extract DNA for exploratory research on genetic biomarkers, MSI, and TMB.

Optional Samples CSF for ctDNA CSF samples are optional for all participants of the study and will be collected for PD and exploratory biomarker assessments. The collection of CSF could be performed by Ommaya reservoir or by lumbar puncture. CSF will be used to assess PD effects of Compound Ia in combination with cobimetinib in brain lesions by investigating the changes in ctDNA.

### Statistical Considerations:

(a) SAMPLE SIZE DETERMINATION: The exact number of participants will depend on the occurrence of DLTs and the number of dose-levels required to determine the MTD and/or the RP2D. The study will enroll a maximum of 120 participants in the dose-escalation stage (60 participants each for Part 1a and Part 1b) and up to approximately 40 participants in up to 4 expansions in Part 2. For FE, an additional cohort of 6-12 participants will be enrolled. The overall maximum number of participants will be 292.

Sample-size justification for the dose expansion Part 2: The sample size of 40 response-evaluable participants per cohort in Part 2 allows to declare futility with 80% chances under the assumption that the true objective response rate (ORR) is 10%, based on the posterior probability for ORR to be below 20% with a 70% confidence level. The futility level of 20% is based on the response rate obtained by vemurafinib in melanoma patients with brain metastases (McArthur et al. 2017). Futility will be assessed after 10, 20, 30, and 40 participants have mature data, respectively. Futility will be concluded if ≤1 of 10 participants, or ≤2 of 20 participants, or ≤4 of 30, or ≤6 of 40 participants have a CR or PR.

Sample size justification for the food cohort: The sample size was determined by practical considerations and is not based on statistical power calculations.

(b) SETS FOR ANALYSES: For purposes of analysis, the following populations are defined in Table 1.

TABLE 1
Analysis Sets:

| Population | Description |
|---|---|
| Safety | All participants enrolled into the study and who |
| | received at least one dose of the study treatment, |
| | whether prematurely withdrawn from the study or not, |
| | will be included in the safety analysis. |
| Response | All participants in the safety population who have a |
| evaluable | baseline tumor evaluation and at least one tumor |
| | evaluation on-treatment. Participants who discontinue |
| | prior to the first on-treatment tumor evaluation due to |
| | clinical progression will be considered as |
| | response-evaluable. |
| Pharmacokinetic | All participants who have received at least one dose of |
| | study treatment and who have data from at least one |
| | postdose sample will be included in the PK analysis |
| | population. Participants will be excluded from the PK |
| | analysis population if they significantly violate the |
| | inclusion or exclusion criteria, deviate significantly |
| | from the protocol, or if data are unavailable or |
| | incomplete which may influence the PK analysis. |
| | Excluded cases will be documented with the reason for |
| | exclusion. All decisions on exclusions from the |
| | analysis will be made prior to database closure. |
| DLT evaluable | Participants are eligible for DLT evaluation if they |
| | experience a DLT after at least one dose of study |
| | treatment or do not experience a DLT having taken a |
| | minimum of 80% of doses expected during the DLT |
| | observation window. Participants who do not fulfill |
| | these requirements and/or who discontinue study |
| | participation prior to completing the DLT assessment |
| | period will be replaced. |

DLT = dose-limiting toxicities; PK = pharmacokinetic.

(c) STATISTICAL ANALYSES: When appropriate, data will be summarized by cohort, dose level, and/or dosing schedules within each part of the study. Subject with leptomeningeal disease may be analysed separately in each part if the number of patient is sufficient. Demography and baseline characteristics (including but not restricted to age, sex, biomarker status, previous therapies, and medical history) will be analyzed using descriptive statistics. Exposure to study medication will be summarized by total duration of study medication, number of cycles started, and cumulative dose using descriptive statistics. Dose interruptions and their reasons will be presented by schedule and dose level. Efficacy Analyses Participants will be assessed for response by the investigators based on the RECIST v1.1 criteria for extracranial lesions and mRECIST-BM for intra-cranial lesions. Participants with missing or no response assessments will be classified as “not evaluable”. Response will need to be confirmed by a second assessment four weeks after the first assessment showing response. Only the confirmed response will be analysed and used for futility analysis. A participant will be considered as “responder” if the best response on treatment is equal to “complete response” (CR), or “partial response” (PR). The ORR will be defined as the proportion of responder out of the number of response-evaluable participants. The disease control rate (DCR) will be defined as the proportion of participants being either responder or in “stable disease” (SD) at least once at the first post-baseline tumor assessment. Duration of response will be calculated for “responder” participants (i.e., best [confirmed] overall response of CR or PR) and will be defined as the time from first occurrence of a documented response until the time of documented disease progression or death from any cause, whichever occurs first. Progression-free survival (PFS) will be defined as the time from first dose of study treatment to the first occurrence of documented disease progression or death from any cause, whichever occurs first (Table 2). For participants who do not have documented progressive disease or death until the last response assessment during the study, PFS will be censored at the day of the last on study tumor assessment. Participants without any post-baseline assessments or with all post-baseline assessments having unknown result/response but known to be alive at the clinical cut off for the analysis will be censored at the date of study treatment initiation plus one day. Response and PFS analyses will be based on the response-evaluable population.

TABLE 2
Efficacy Statistical Analysis Methods:

| Endpoint | Statistical Analysis Methods |
|---|---|
| ORR | Exact 95% confidence intervals (CIs) using the |
| | Clopper-Pearson method for ORR will be provided |
| DCR | Disease control rate will be summarized using |
| | relative frequencies with CIs |
| DOR, PFS | Duration of response and PFS are based on the |
| | timepoint response assessments and will be |
| | analyzed using Kaplan-Meyer methods |

DCR = disease control rate; DOR = duration of response; PFS = progression-free survival; ORR = objective response rate.

Safety Analyses: All safety parameters listed in Table 3 will be analyzed using descriptive statistics, presented in tables and/or graphics. Safety will be assessed through summaries of DLTs (for Part 1), AEs, changes in laboratory test results, changes in vital signs, ECGs and LVEF, and their respective relation to study treatment exposure.

TABLE 3
Safety Statistical Analysis Methods:

| Endpoint | Statistical Analysis Methods |
|---|---|
| Adverse events | The original terms recorded on the eCRF by the Investigator |
| | for AEs will be coded. AEs will be summarized by mapped |
| | term and appropriate thesaurus level |
| Nature and | DLT events will be presented by individual listings. |
| frequency of DLTs | A DLT will be defined as an AE or clinically significant |
| | abnormal laboratory value not considered by the Investigator to |
| | be attributable to another clearly identifiable cause that occurs |
| | during the DLT observation window and meets the criteria listed |
| | below. |
| | All AEs, including DLTs, will be graded according to National |
| | Cancer Institute (NCI) Common Terminology Criteria for |
| | Adverse Events (CTCAE) v5.0 unless otherwise indicated. |
| | DLTs will be treated according to clinical practice and will be |
| | monitored through their resolution. |
| | Note: AEs that are attributed by the Investigator to another |
| | identifiable cause (e.g., documented disease progression, |
| | concomitant medication, or preexisting medical condition) are |
| | not considered a DLT. |
| | Dose-Limiting Toxicity Criteria |
| | Any Grade ≥3 AE not considered by the Investigator to be |
| | attributable to another clearly identifiable cause, with the |
| | following exceptions: |
| | Grade 3 neutropenia that is not accompanied by temperature |
| | elevation (oral or tympanic temperature of ≥100.4° F. |
| | [38° C.]) and improves to Grade ≤2 within one week. |
| | Grade 3 thrombocytopenia that does not result in Grade ≥2 |
| | bleeding and improves to Grade ≤2 within one week. |
| | Grade 3 nausea, vomiting, or diarrhea that resolves in ≤3 |
| | days with supportive medications. |
| | Grade 3 rash that resolves to Grade ≤2 within 7 days with |
| | appropriate supportive care. |
| | Grade 3 fatigue that resolves to Grade ≤2 within 7 days. |
| | Grade 3 arthralgia that can be adequately managed with |
| | supportive care or that resolves to Grade ≤2 within 7 days. |
| | Grade 3 fever (as defined by >40° C.) for ≤24 hours. |
| | Grade 3 alopecia. |
| | Grade 3 laboratory abnormality that is asymptomatic and |
| | deemed by the investigator not to be clinically significant. |
| | Grade 3 hypertension which can be controlled within 3 days |
| | to grade ≤2 with antihypertensive medication. |
| | Severe hepatotoxicity defined as: |
| | Grade ≥3 elevation of total bilirubin or ALT/AST or ALP |
| | lasting >7 days, with the following exception: for |
| | participants with Grade 2 hepatic transaminase or ALP at |
| | baseline as a result of liver or bone metastases, hepatic |
| | transaminase or ALP ≤10 × upper limit of normal (ULN) |
| | will not be considered a DLT. |
| | Any increase in hepatic transaminase (ALT or AST) >3 × |
| | ULN in combination with either an increase in total |
| | bilirubin >2 × ULN (of which ≥35% is direct bilirubin) or |
| | clinical jaundice, in the absence of cholestasis or other |
| | contributory factors (e.g., worsening of metastatic disease, |
| | concomitant exposure to known hepatotoxic agent, or documented |
| | infectious etiology). This is suggestive of potential drug- |
| | induced liver injury (according to Hy's Law). For |
| | participants with liver metastases, ALT or AST >5 × ULN |
| | value in combination with total bilirubin > 2 x ULN (of |
| | which ≥35% is direct bilirubin) or clinical jaundice, should |
| | be used to define severe hepatotoxicity. |
| | Additionally, the following is defined as a DLT: |
| | Failure to recover from any Compound I-related toxicity that |
| | results in a dose delay of more than 7 days (any regimen) is |
| | defined as a DLT. |
| | QTcF shortening defined either by magnitude (≤340 ms |
| | or >15% decrease from the baseline value), or due to clear |
| | association with relevant symptoms e.g., dizziness or pre- |
| | syncope. |
| | Participants who experience a DLT during the DLT |
| | observation window may continue study treatment at their |
| | current or a lower dose once the toxicity has improved to |
| | Grade ≤1 within 28 days. |
| Clinical laboratory | All clinical laboratory data will be stored in the database in |
| tests | the units in which they were reported. Laboratory test values |
| | will be presented in International System of Units (SI units; |
| | Systeme International d'Unites) by individual listings with |
| | flagging of abnormal results. Summary tables of change from |
| | baseline over time will be displayed. Shifts in NCI CTCAE |
| | grades v5.0 from baseline to the worst grade observed during |
| | treatment will be presented for selected laboratory |
| | parameters. |
| Vital signs | Vital sign data will be presented by individual listings with |
| | flagging of values outside the normal ranges and flagging of |
| | abnormalities. In addition, tabular summaries will be used, as |
| | appropriate. |
| Concomitant | The original terms recorded on the participants' eCRF by the |
| medications | Investigator for concomitant medications will be standardized |
| | by the Sponsor by utilizing a mapped term and appropriate |
| | drug dictionary level. Concomitant medications will be |
| | presented using appropriate outputs. |
| ECHO/MUGA & | ECHO/MUGA and ECG data will be presented by individual |
| ECG data analysis | listings. In addition, tabular summaries will be used, as |
| | appropriate. |

AE = adverse event; DLT = dose-limiting toxicities; ECG = electrocardiogram; ECHO = echocardiogram; eCRF = electronic case report form; MUGA = multiple-gated acquisition; NCI CTCAE-National Cancer Institute Common Terminology Criteria for Adverse Events.

### Pharmacokinetic Analyses:

PK parameters for Compound Ia administered alone and in combination with cobimetinib will be derived from the plasma concentrations and will be listed and summarized using descriptive statistics (such as mean, standard deviation, coefficient of variation, median, minimum, and maximum). Actual sampling times are presented in Table 13. The PK parameters will include but are not limited to area under the curve (AUC), maximum concentration (Cₘₐₓ), time of maximum concentration observed (Tₘₐₓ) and, apparent oral clearance (CL/F), volume of distribution at steady-state (Vₛₛ/F), and terminal half-life (t½). Estimation of PK parameters may be performed using standard non-compartmental methods and/or population PK modeling. If data allow, other methods may be used for data analysis, e.g., PK and PD data may be used to develop a population PK/PD model. Individual and mean plasma Compound Ia concentration versus time data will be tabulated and plotted by dose levels. Graphical displays of PK data may also be provided. The linearity of PK (AUC and Cₘₐₓ) will be investigated. To assess the FE on the PK of Compound Ia, PK parameters (such as AUC, Cₘₐₓ, Tₘₐₓ and t_{1/2}) will be analyzed following oral administration of Compound Ia while either fed (high-fat meal) or fasted for a minimum of 6 participants. Estimation of PK parameters may be performed using standard non-compartmental methods and/or population PK modeling. Furthermore, Compound Ia CSF concentrations (where applicable) might be presented by listings and descriptive summary statistics (such as mean, standard deviation, coefficient of variation, median, minimum, and maximum). If data allow, other methods may be used for data analysis, e.g., PK and PD data may be used to develop a population PK/PD model. For participants receiving cobimetinib, PK parameters for cobimetinib (such as but not limited to AUC, Cₘₐₓ, Tₘₐₓ and t_{1/2}) will be listed and compared with historical exposures observed in previous studies with cobimetinib to evaluate the induction potential of Compound Ia. Estimation of PK parameters may be performed using standard non-compartmental methods and/or population PK modeling. In addition, the relationship between drug exposure (Compound Ia or cobimetinib), safety, PD, and efficacy endpoints will be explored for establishing an exposure-response relationship. Additional PK analyses for Compound Ia (and Compound Ia metabolite(s), if available and appropriate) or cobimetinib will be conducted as appropriate on the basis of available data. Results of PK and/or any exposure-response relationship may be reported outside the clinical study report.

### Pharmacodynamic Analyses:

All PD parameters will be presented by listings and descriptive summary statistics separately by group or cohorts. Descriptive statistics will be used to summarize peripheral blood and tumor PD markers. Absolute and percentage change from baseline will be calculated for the PD markers. Graphical techniques will be employed to better understand the relationship of the PD markers with dose and time.

### Pharmacokinetic/Pharmacodynamic Relationships:

Correlations between PK parameters, PD markers, and clinical response will be assessed through data tabulations and graphical techniques. If data allow, other methods may be used for data analysis, e.g., PK and PD data may be used to develop a population PK/PD model.

### Other Analyses:

Endogenous CYP3A Activity Marker Analysis: For the analysis of CYP3A activity during Compound Ia administration, the plasma concentrations of 4βHC, TC and/or 4αHC will be determined and the percent change from baseline of 4βHC with and without normalization by TC and 4αHC will be derived. If data allow, other exploratory methods may be used for data analysis, e.g., PK and CYP3A activity data may be used to develop a population PK/CYP3A activity model.
[18F]-FDG-PET Imaging: For the analysis of FDG-PET data, participants with baseline and at least one post-baseline scan will be considered evaluable. Uptake of [18F]-FDG-PET performed at baseline and on-treatment time-points will be summarized descriptively. Change and percentage of change of FDG-PET parameters (such as SUVₘₐₓ, SUVₘₑₐₙ, total lesion glycolysis, etc.) from baseline will be described and evaluated as evidence of response to treatment.

### Interim Analyses:

In Part 2, a futility analysis will be conducted after 10, 20, and 30 participants of each expansion cohort, respectively.

### Definition of Adverse Events:

According to the E2A ICH guideline for Good Clinical Practice, an adverse event (AE) is any untoward medical occurrence in a participant or clinical investigation participant administered a pharmaceutical product and which does not necessarily have to have a causal relationship with this treatment. An AE can therefore be: Any unfavorable and unintended sign (including an abnormal laboratory finding), symptom, or disease temporally associated with the use of a medicinal product, whether or not considered related to the medicinal product.

### Events Meeting the AE Definition:

Deterioration in a laboratory value (hematology, clinical chemistry, or urinalysis) or other clinical test (e.g., ECG, X-ray) that is associated with symptoms or leads to a change in study treatment or concomitant treatment or discontinuation from study treatment.
Exacerbation of a chronic or intermittent preexisting condition, including either an increase in frequency and/or intensity of the condition.
New conditions detected or diagnosed after study treatment administration even though it may have been present before the start of the study.
AEs that are related to a protocol-mandated intervention, including those that occur prior to assignment of study treatment (e.g., screening invasive procedures such as biopsies). “Lack of efficacy” or “failure of expected pharmacological action” per se will not be reported as an AE or serious AE (SAE) unless the progression is unexpectedly accelerated and not in line with the natural history of the disease. If the “Lack of efficacy” would not require safety reporting such instances will be captured in the efficacy assessments. However, the signs, symptoms, and/or clinical sequelae resulting from lack of efficacy will be reported as AE or SAE if they fulfill the definition of an AE or SAE.

### Events NOT Meeting the AE Definition:

Any clinically significant abnormal laboratory findings or other abnormal safety assessments which are associated with the underlying disease, unless judged by the Investigator to be more severe than expected for the participant's condition.
The disease/disorder being studied or expected progression, signs, or symptoms of the disease/disorder being studied, unless more severe than expected for the participant's condition. Medical or surgical procedure (e.g., endoscopy, appendectomy): the condition that leads to the procedure is an AE.
Situations where an untoward medical occurrence did not occur (social and/or convenience admission to a hospital).
Anticipated day-to-day fluctuations of preexisting disease(s) or condition(s) present or detected at the start of the study that do not worsen.

DEFINITION OF SERIOUS ADVERSE EVENTS If an event is not an AE per definition above, then it cannot be an SAE even if serious conditions are met (e.g., hospitalization for signs/symptoms of the disease under study, death due to progression of disease).

An SAE is defined as any untoward medical occurrence that at any dose:
Results in death.
Is life-threatening.
   The term “life-threatening” in the definition of “serious” refers to an event in which the participant was at risk of death at the time of the event; it does not refer to an event which hypothetically might have caused death if it was more severe.
Requires inpatient hospitalization or prolongation of existing hospitalization. In general, hospitalization signifies that the participant has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious.
   Hospitalization for elective treatment of a preexisting condition that did not worsen from baseline is not considered an AE.
Results in persistent or significant disability/incapacity
   Disability means substantial disruption of the participant's ability to conduct normal life functions.
   This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.
Is a congenital anomaly/birth defect.
Other significant events: Medical or scientific judgement should be exercised in deciding whether SAE reporting is appropriate in other situations such as important medical events that may not be immediately life-threatening or result in death or hospitalization but may jeopardize the participant or may require medical or surgical intervention to prevent one of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

### Assessment of Severity:

The terms “severe” and “serious” are not synonymous. Severity refers to the intensity of an AE (rated as mild, moderate, or severe, or according to a pre-defined grading criteria [e.g., National Cancer Institute Common Terminology Criteria for Adverse Events [NCI CTCAE] criteria); the event itself may be of relatively minor medical significance (such as severe headache without any further findings). Severity and seriousness need to be independently assessed for each AE recorded. The AE severity grading scale for the NCI CTCAE (v5.0) will be used for assessing AE severity. Table 4 will be used for assessing severity for AEs that are not specifically listed in the NCI CTCAE.

TABLE 4
Adverse Event Severity Grading Scale:

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention |
| | indicated; or limiting age-appropriate instrumental activities |
| | of daily living^{a} |
| 3 | Severe or medically significant, but not immediately |
| | life-threatening; hospitalization or prolongation of |
| | hospitalization indicated; disabling; or limiting |
| | self-care activities of daily living^{b} |
| 4 | Life-threatening consequences or urgent intervention |
| | indicated^{d} |
| 5 | Death related to adverse event^{d} |

NCI CTCAE = National Cancer Institute Common Terminology Criteria for Adverse Events.
Note:
Based on the NCI CTCAE (v5.0).
^{a}Instrumental activities of daily living refer to preparing meals, shopping for groceries or clothes, using the telephone, managing money, etc.
^{b}Examples of self-care activities of daily living include bathing, dressing and undressing, feeding one's self, using the toilet, and taking medications, as performed by patients who are not bedridden.
c If an event is assessed as a “significant medical event,” it must be reported as a serious adverse event per the definition further below.
^{d}Grade 4 and 5 events must be reported as serious adverse events per the definition of serious adverse events above. Grade 4 laboratory abnormalities would only be reported as SAEs if these meets one or more of the conditions outlined further below.

### Assessment of Causality:

Investigators should use their knowledge of the participant, the circumstances surrounding the event, and an evaluation of any potential alternative causes to determine whether or not an AE is considered to be related to the study treatment, indicating “yes” or “no” accordingly. The following guidance should be taken into consideration:
Temporal relationship of event onset to the initiation of study treatment.
Course of the event, considering especially the effects of dose-reduction, discontinuation of study treatment, or reintroduction of study treatment.
Known association of the event with the study treatment or with similar treatments.
Known association of the event with the disease under study.
Presence of risk factors in the participant or use of concomitant medications known to increase the occurrence of the event.
Presence of non-treatment-related factors that are known to be associated with the occurrence of the event.

For participant receiving combination therapy, causality will be assessed individually for each protocol-mandated therapy.

### Measurability of Tumor at Baseline

Definitions: At baseline, tumor lesions/lymph nodes will be categorized as measurable or non-measurable as described below.

Measurable tumor lesions—Tumor lesions: Tumor lesions must be accurately measured in at least one dimension (longest diameter in the plane of measurement is to be recorded) with a minimum size as follows:
10 mm by computed tomography (CT) or magnetic resonance imaging (MRI) scan (CT/MRI scan slice thickness/interval no greater than 5 mm)
10-mm caliper measurement by clinical examination (lesions that cannot be accurately measured with calipers should be recorded as non-measurable)
20 mm by chest X-ray

Measurable tumor lesions—Malignant lymph nodes To be considered pathologically enlarged and measurable, a lymph node must be ≥15 mm in the short axis when assessed by CT scan (CT scan slice thickness is recommended to be no greater than 5 mm). At baseline and follow up, only the short axis will be measured and followed. See also notes below on “Baseline Documentation of Target and Non-Target Lesions” for information on lymph node measurement.

Non-Measurable tumor lesions: Non-measurable tumor lesions encompass small lesions (longest diameter <10 mm or pathological lymph nodes with ≥10 to ≤15 mm short axis) as well as truly non-measurable lesions. Lesions considered truly non-measurable include leptomeningeal disease, ascites, pleural or pericardial effusion, inflammatory breast disease, lymphangitic involvement of skin or lung, peritoneal spread, and abdominal mass/abdominal organomegaly identified by physical examination that is not measurable by reproducible imaging techniques.

Special considerations regarding lesion measurability: Bone lesions, cystic lesions, and lesions previously treated with local therapy require particular comment, as outlined below.

Bone lesions; Bone scan, positron emission tomography (PET) scan, or plain films are not considered adequate imaging techniques for measuring bone lesions. However, these techniques can be used to confirm the presence or disappearance of bone lesions. Lytic bone lesions or mixed lytic-blastic lesions, with identifiable soft tissue components, that can be evaluated by cross-sectional imaging techniques, such as CT or MRI, can be considered as measurable lesions if the soft tissue component meets the definition of measurability described above. Blastic bone lesions are non-measurable.

Cystic lesions: Lesions that meet the criteria for radiographically defined simple cysts should not be considered as malignant lesions (neither measurable nor non-measurable) because they are, by definition, simple cysts. Cystic lesions thought to represent cystic metastases can be considered measurable lesions if they meet the definition of measurability described above. However, if non-cystic lesions are present in the same patient, these are preferred for selection as target lesions.

Lesions with prior local treatment: Tumor lesions situated in a previously irradiated area, or in an area subjected to other loco-regional therapy, are usually not considered measurable unless there has been demonstrated progression in the lesion. Study protocols should detail the conditions under which such lesions would be considered measurable.

### Target Lesions: Specifications by Methods of Measurements:

Measurement of lesions: All measurements should be recorded in metric notation, using calipers if clinically assessed. All baseline evaluations should be performed as close as possible to the treatment start and never more than 4 weeks before the beginning of the treatment.

Method of assessment: The same method of assessment and the same technique should be used to characterize each identified and reported lesion at baseline and during the study. Imaging-based evaluation should always be the preferred option.

Clinical lesions: Clinical lesions will only be considered measurable when they are superficial and ≥10 mm in diameter as assessed using calipers (e.g., skin nodules). For the case of clinical lesions, documentation by color photography, including a ruler to estimate the size of the lesion, is required.

Chest X-Ray: Chest CT is preferred over chest X-Ray, particularly when progression is an important endpoint, because CT is more sensitive than X-Ray, particularly in identifying new lesions. However, lesions on chest X-Ray may be considered measurable if they are clearly defined and surrounded by aerated lung.

CT, MRI: CT is the best currently available and reproducible method to measure lesions selected for response assessment. This guideline has defined measurability of lesions on CT scan on the basis of the assumption that CT slice thickness is 5 mm or less. When CT scans have slice thickness greater than 5 mm, the minimum size for a measurable lesion should be twice the slice thickness. MRI is also acceptable. If, prior to enrollment, it is known that a patient is unable to undergo CT scans with intravenous (IV) contrast because of allergy or renal insufficiency, the decision as to whether a non-contrast CT or MRI (without IV contrast) will be used to evaluate the patient at baseline and during the study should be guided by the tumor type under investigation and the anatomic location of the disease. For patients who develop contraindications to contrast after baseline contrast CT is done, the decision as to whether non-contrast CT or MRI (enhanced or non-enhanced) will be performed should also be based on the tumor type and the anatomic location of the disease and should be optimized to allow for comparison with the prior studies if possible. Each case should be discussed with the radiologist to determine if the substitution of these other approaches is possible and, if not, the patient should be considered not evaluable from that point forward. Care must be taken in measurement of target lesions on a different modality and interpretation of non-target disease or new lesions because the same lesion may appear to have a different size using a new modality.

Ultrasound: Ultrasound is not useful in assessment of lesion size and should not be used as a method of measurement.

Endoscopy, laparoscopy, tumor markers, cytology, histology: The utilization of these techniques for objective tumor evaluation cannot generally be advised.

### Tumor Response Evaluation—Assessment of Overall Tumor Burden and Measurable Disease:

To assess objective response or future progression, it is necessary to estimate the overall tumor burden at baseline and use this as a comparator for subsequent measurements. Measurable disease is defined by the presence of at least one measurable lesion, as detailed above.

### Tumor Response Evaluation—Baseline Documentation of Target and Non-Target Lesions:

When more than one measurable lesion is present at baseline, all lesions, up to a maximum of five lesions total and a maximum of two lesions per organ, representative of all involved organs should be identified as target lesions and will be recorded and measured at baseline. This means that, for instances in which patients have only one or two organ sites involved, a maximum of two lesions (one site) and four lesions (two sites), respectively, will be recorded. Other lesions (albeit measurable) in those organs will be recorded as non-measurable lesions (even if the size is >10 mm by CT scan). Target lesions should be selected on the basis of their size (lesions with the longest diameter) and be representative of all involved organs, but, in addition, the lesions should lend themselves to reproducible repeated measurements. It may be the case that, on occasion, the largest lesion does not lend itself to reproducible measurement, in which circumstance the next largest lesion that can be measured reproducibly should be selected. Lymph nodes merit special mention because they are normal anatomical structures that may be visible by imaging even if not involved by tumor. As noted above, pathological nodes that are defined as measurable and may be identified as target lesions must meet the criterion of a short axis of ≥15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT this is almost always the axial plane; for MRI the plane of acquisition may be axial, sagittal, or coronal). The smaller of these measures is the short axis. For example, an abdominal node that is reported as being 20 mm×30 mm has a short axis of 20 mm and qualifies as a malignant, measurable node. In this example, 20 mm should be recorded as the node measurement. All other pathological nodes (those with short axis ≥10 mm but ≤15 mm) should be considered non-target lesions. Nodes that have a short axis of ≤10 mm are considered non-pathological and should not be recorded or followed. A sum of the diameters (longest for non-nodal lesions, short axis for nodal lesions) for all target lesions will be calculated and reported as the baseline sum of diameters. If lymph nodes are to be included in the sum, then, as noted above, only the short axis is added into the sum. The baseline sum of diameters will be used as a reference to further characterize any objective tumor regression in the measurable dimension of the disease. All other lesions or sites of disease, including pathological lymph nodes, should be identified as non-target lesions and should also be recorded at baseline. Measurements are not required and these lesions should be followed as “present,” “absent,” or in rare cases “unequivocal progression”. In addition, it is possible to record multiple non-target lesions involving the same organ as a single item on the eCRF (e.g., “multiple enlarged pelvic lymph nodes” or “multiple liver metastases”).

RESPONSE CRITERIA—Evaluation of target lesions: This section provides the definitions of the criteria used to determine objective tumor response for target lesions.
CR (complete response): Disappearance of all target lesions
   Any pathological lymph nodes (whether target or non-target) must have reduction in short axis to <10 mm.
PR (partial response): At least a 30% decrease in the sum of diameters of target lesions, taking as reference the baseline sum of diameters.
Progressive disease: At least a 20% increase in the sum of diameters of target lesions, taking as reference the smallest sum on study (nadir), including baseline.
   In addition to the relative increase of 20%, the sum must also demonstrate an absolute increase of at least 5 mm.
   The appearance of one or more new lesions is also considered progression.
SD (stable disease): Neither sufficient shrinkage to qualify for PR nor sufficient increase to qualify for progressive disease, taking as reference the smallest sum on study.

Special notes on the assessment of target lesions: Lymph nodes identified as target lesions should always have the actual short axis measurement recorded (measured in the same anatomical plane as the baseline examination), even if the nodes regress to <10 mm on study. This means that when lymph nodes are included as target lesions, the sum of lesions may not be zero even if CR criteria are met, because a normal lymph node is defined as having a short axis of <10 mm.

Target lesions that become too small to measure: During the study, all lesions (nodal and non-nodal) recorded at baseline should have their actual measurements recorded at each subsequent evaluation, even when very small (e.g., 2 mm). However, sometimes lesions or lymph nodes that are recorded as target lesions at baseline become so faint on the CT scan that the radiologist may not feel comfortable assigning an exact measure and may report them as being too small to measure. When this occurs, it is important that a value be recorded on the eCRF, as follows: If it is the opinion of the radiologist that the lesion has likely disappeared, the measurement should be recorded as 0 mm. If the lesion is believed to be present and is faintly seen but is too small to measure, a default value of 5 mm should be assigned and BML (below measurable limit) should be ticked. (Note: It is less likely that this rule will be used for lymph nodes since they usually have a definable size when normal and are frequently surrounded by fat such as in the retroperitoneum; however, if a lymph node is believed to be present and is faintly seen but is too small to measure, a default value of 5 mm should be assigned in this circumstance as well and BML should also be ticked). To reiterate: If the radiologist is able to provide an actual measure, this measurement should be recorded, even if it is below 5 mm, and, in that case, BML should not be ticked. Lesions that split or coalesce on treatment When non-nodal lesions fragment, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximum longest diameter for the coalesced lesion.

Evaluation of Non-Target Lesions: This section provides the definitions of the criteria used to determine the tumor response for the group of non-target lesions. Whereas some non-target lesions may actually be measurable, they need not be measured and instead should be assessed only qualitatively at the timepoints specified in the protocol.
CR: Disappearance of all non-target lesions. All lymph nodes must be non-pathological in size (<10 mm short axis).
Non-CR/Non-progressive disease: Persistence of one or more non-target lesions.
Progressive disease: Unequivocal progression of existing non-target lesions. The appearance of one or more new lesions is also considered progression.

Special notes on assessment of progression of non-target disease:
When the patient also has measurable disease: In this setting, to achieve unequivocal progression on the basis of the non-target disease, there must be an overall level of substantial worsening in non-target disease in a magnitude that, even in the presence of SD or PR in target disease, the overall tumor burden has increased sufficiently to merit discontinuation of therapy. A modest increase in the size of one or more non-target lesions is usually not sufficient to qualify for unequivocal progression status. The designation of overall progression solely on the basis of change in non-target disease in the face of SD or PR of target disease will therefore be extremely rare.
When the patient has only non-measurable disease: This circumstance arises in some Phase III trials when it is not a criterion of study entry to have measurable disease. The same general concepts apply here as noted above; however, in this instance, there is no measurable disease assessment to factor into the interpretation of an increase in non-measurable disease burden. Because worsening in non-target disease cannot be easily quantified (by definition: if all lesions are truly non-measurable), a useful test that can be applied when assessing patients for unequivocal progression is to consider if the increase in overall disease burden on the basis of the change in non-measurable disease is comparable in magnitude to the increase that would be required to declare progressive disease for measurable disease, that is, an increase in tumor burden representing an additional 73% increase in volume (which is equivalent to a 20% increase in diameter in a measurable lesion). Examples include an increase in a pleural effusion from “trace” to “large” or an increase in lymphangitic disease from localized to widespread. Examples may be described in protocols as “sufficient to require a change in therapy”. If unequivocal progression is seen, the patient should be considered to have had overall progressive disease at that point. Though it would be ideal to have objective criteria to apply to non-measurable disease, the very nature of that disease makes it impossible to do so; therefore, the increase must be substantial.
New lesions: The appearance of new malignant lesions denotes disease progression; therefore, some comments on detection of new lesions are important. There are no specific criteria for the identification of new radiographic lesions; however, the finding of a new lesion should be unequivocal, that is, not attributable to differences in scanning technique, change in imaging modality, or findings thought to represent something other than tumor (e.g., some “new” bone lesions may be simply healing or flare of preexisting lesions). This is particularly important when the patient's baseline lesions show PR or CR (e.g., necrosis of a liver lesion may be reported on a CT scan report as a “new” cystic lesion, which it is not). A lesion identified during the study in an anatomical location that was not scanned at baseline is considered a new lesion and will indicate disease progression. If a new lesion is equivocal (e.g., because of its small size) continued therapy and follow-up evaluation will clarify if it represents truly new disease. If repeat scans confirm there is definitely a new lesion, then progression should be declared using the date of the initial scan.

### Evaluation of Response:

Timepoint response (overall response): It is assumed that at each protocol-specified timepoint, a response assessment occurs. Table 5 provides a summary of the overall response status calculation at each timepoint for patients who have measurable disease at baseline. When patients have non-measurable (therefore non-target) disease only, Table 6 is to be used.

TABLE 5
Timepoint Response: Patients with Target Lesions
(with or without Non-Target Lesions)

| | Non-Target | | Overall |
|---|---|---|---|
| Target Lesions | Lesions | New Lesions | Response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or no | PD |
| Any | PD | Yes or no | PD |
| Any | Any | Yes | PD |

CR = complete response; NE = not evaluable; PD = progressive disease; PR = partial response; SD = stable disease.

TABLE 6
Timepoint Response: Patients with Non-Target Lesions Only

| | | Overall |
|---|---|---|
| Non-Target Lesions | New Lesions | Response |
| CR | No | CR |
| Non-CR/non-PD | No | Non-CR/non-PD |
| Not all evaluated | No | NE |
| Unequivocal PD | Yes or No | PD |
| Any | Yes | PD |

CR = complete response; NE = not evaluable; PD = progressive disease.

Missing assessments and not evaluable designation: When no imaging/measurement is done at all at a particular timepoint, the patient is not evaluable at that timepoint. If only a subset of lesion measurements are made at an assessment, usually the case is also considered not evaluable at that timepoint unless a convincing argument can be made that the contribution of the individual missing lesion(s) would not change the assigned timepoint response. This would be most likely to happen in the case of progressive disease (e.g., if a patient had a baseline sum of 50 mm with three measured lesions and during the study only two lesions were assessed, but those gave a sum of 80 mm, the patient will have achieved progressive disease status, regardless of the contribution of the missing lesion). If one or more target lesions were not assessed either because the scan was not done or because the scan could not be assessed due to poor image quality or obstructed view, the response for target lesions should be “unable to assess” because the patient is not evaluable. Similarly, if one or more non-target lesions are not assessed, the response for non-target lesions should be “unable to assess”, except where there is clear progression. Overall response would be “unable to assess” if either the target response or the non-target response is “unable to assess”, except where this is clear evidence of progression, as this equates with the case being not evaluable at that timepoint.

TABLE 7
Best Overall Response when Confirmation is Required

| Overall | Overall | |
|---|---|---|
| Response | Response | |
| at First | at Subsequent | |
| Timepoint | Timepoint | Best Overall Response |
| CR | CR | CR |
| CR | PR | SD, PD, or PR |
| CR | SD | SD, provided minimum duration for |
| | | SD was met; otherwise, PD |
| CR | PD | SD, provided minimum duration for |
| | | SD was met; otherwise, PD |
| CR | NE | SD, provided minimum duration for |
| | | SD was met; otherwise, NE |
| PR | CR | PR |
| PR | PR | PR |
| PR | SD | SD |
| PR | PD | SD, provided minimum duration for |
| | | SD was met; otherwise, PD |
| PR | NE | SD, provided minimum duration for |
| | | SD was met; otherwise, PD |
| NE | NE | NE |

CR = complete response; NE = not evaluable; PD = progressive disease; PR = partial response; SD = stable disease.

Special notes on response assessment: When nodal disease is included in the sum of target lesions and the nodes decrease to “normal” size (<10 mm), they may still have a measurement reported on scans. This measurement should be recorded even though the nodes are normal in order not to overstate progression should it be based on increase in size of the nodes. As noted earlier, this means that patients with CR may not have a total sum of “zero” on the eCRF. Patients with a global deterioration of health status requiring discontinuation of treatment without objective evidence of disease progression at that time should be reported as “symptomatic deterioration.” Every effort should be made to document objective progression even after discontinuation of treatment. Symptomatic deterioration is not a descriptor of an objective response; it is a reason for stopping study therapy. The objective response status of such patients is to be determined by evaluation of target and non-target disease as shown in Table 5 to Table 7. For equivocal findings of progression (e.g., very small and uncertain new lesions; cystic changes, or necrosis in existing lesions), treatment may continue until the next scheduled assessment. If at the next scheduled assessment, progression is confirmed, the date of progression should be the earlier date when progression was suspected. In studies for which patients with advanced disease are eligible (i.e., primary disease still or partially present), the primary tumor should also be captured as a target or non-target lesion, as appropriate. This is to avoid an incorrect assessment of complete response if the primary tumor is still present but not evaluated as a target or non-target lesion.

Modified Response Assessment in Neuro-Oncology for Brain Metastases (mRECIST-BM):

RANO-BM criteria are referred to as mRECIST-BM criteria in this protocol. mRECIST-BM CRITERIA Similar to Response Evaluation Criteria in Solid Tumors (RECIST) v1.1, definitions for radiographical response will be based on unidimensional measurements.

DEFINITIONS Measurable disease is defined as a contrast-enhancing lesion that can be accurately measured in at least one dimension, with a minimum size of 10 mm, and is visible on two or more axial slices that are preferably 5 mm or less apart with 0 mm skip (and ideally ≤1.5 mm apart with 0 mm skip). Additionally, although the longest diameter in the plane of measurement is to be recorded, the diameter perpendicular to the longest diameter in the plane of measurement should be at least 5 mm for the lesion to be considered measurable. If the magnetic resonance imaging (MRI) is performed with thicker slices, the size of the measurable lesion at baseline should be at least double the slice thickness. Interslice gaps, if present, should also be considered in the determination of the minimum size of measurable lesions at baseline.

Measurement of a tumor around a cyst or surgical cavity is a particularly di cult challenge. Generally, such lesions should be considered non-measurable unless there is a nodular component that measures 10 mm or more in longest diameter and 5 mm or more in the perpendicular plane. The cystic or surgical cavity should not be measured for the determination of a response. Non-measurable disease includes all other lesions, including lesions with longest dimension less than 10 mm, lesions with borders that cannot be reproducibly measured, dural metastases, bony skull metastases, cystic-only lesions, and leptomeningeal disease. Patients with non-measurable disease can still be included in trials where response is not the primary endpoint (e.g., in trials with progression-free survival, overall survival, or other primary endpoints). For studies in which CNS objective response is the primary endpoint, we generally recommend a cutoff of 10 mm to limit the study to measurable disease. For investigators who choose to lower the minimum size limit of measurable disease to 5 mm, we strongly recommend MRI imaging with 1.5 mm slice thickness or less. Complete response and unequivocal progressive disease can probably be interpreted even with lesions as small as 5 mm. However, measurement of small changes, such as the minimum 20% increase in longest diameter to determine progressive disease or the minimum 30% decrease in longest diameter to determine partial response, might not be robust or reproducible. With the intrinsic uncertainty of measurements of small lesions, any lesion less than 10 mm in longest diameter should be regarded as unchanged from baseline unless there is a minimum 3 mm change in the measured longest diameter. The decision to include patients with multiple lesions with a sum diameter of 10 mm or more but of which the largest lesion measures less than 10 mm should be taken with caution if objective response is the primary endpoint. If such patients are included, response should be assessed using the sum of the longest diameters of the lesions, and the response criteria should be clearly delineated in the protocol. Thin-section MRI imaging with 1.5 mm or thinner slice thickness would be necessary in this setting.

### Methods of Measurement:

The same method of assessment and the same technique should be used to characterize each identified and reported lesion at baseline and during follow-up. Consistent use of imaging techniques across all imaging timepoints is important to ensure that the assessment of interval appearance, disappearance of lesions, or change in size is not affected by scan parameters such as slice thickness. Use of thin section imaging is particularly important for the assessment of lesions less than 10 mm in longest diameter or small changes in lesion size, or both. Gadolinium-enhanced MRI is the most sensitive and reproducible method available to measure CNS lesions selected for response assessment (Schellinger et al. 1999, Sze et al. 1990). MRI is strongly encouraged as the default standard imaging technique, although CT with and without contrast could be considered in specific circumstances (e.g., countries with limited medical resources or contraindication for MRI).

### Tumor-Response Assessment:

Only patients with measurable CNS disease at baseline should be included in protocols where objective CNS tumor response is the primary endpoint. For studies in which objective response is not the primary endpoint, the protocol must specify prospectively whether entry is restricted to those with measurable disease or if patients with non-measurable disease are also eligible. Assignment of CNS response is independent of systemic disease response. CNS lesions are to be assessed according to mRECIST-BM criteria, whereas non-CNS lesions would most typically be assessed according to RECIST 1.1 criteria. Generally, CNS lesions should initially be re-assessed by MRI at protocol-specified intervals 6-12 weeks apart, although there might be specific circumstances in which longer (or shorter) intervals are desirable. For patients who remain stable for extended periods of time, a longer interval between scans might be appropriate. All baseline assessments should be done as close as possible to the treatment start and no more than 4 weeks before the beginning of treatment. For previously treated lesions, we recommend documentation of how each lesion was previously treated (e.g., stereotactic radio-surgery, whole brain radiotherapy, surgical resection). When more than one measurable lesion in the CNS is present at baseline, all lesions up to a maximum of five CNS lesions should be identified as target lesions and will be recorded and measured at baseline. All measurements should be recorded in metric notation. Target lesions should be selected on the basis of their size (longest diameter) and as those that can be measured reproducibly. For patients with recurrent disease who have multiple lesions, of which only one or two are increasing in size, the enlarging lesions should be prioritized as target lesions for the response assessment. Lesions with prior local treatment (i.e., stereotactic radiosurgery or surgical resection) can be considered measurable if progression has occurred since the time of local treatment. However, careful consideration should be given to lesions previously treated with stereotactic radiosurgery, in view of the possibility of treatment effect. Whether such lesions can be considered measured should be specified prospectively in the clinical protocol. If lesions not previously treated with local therapies are present, these are preferred for selection as target lesions. A sum of the diameters for all target lesions will be calculated and reported as the baseline sum of longest diameters. All other CNS lesions should be identified as non-target lesions and should also be recorded at baseline. Measurements are not required and these lesions should be classified as present, absent, or unequivocal progression, and follow up.

### Definition of Best Overall CNS Response:

Best overall CNS response is a composite of radiographical CNS target and non-target lesion responses (Table 8). For non-randomized trial in which CNS response is the primary endpoint, confirmation of partial response or complete response at least 4 weeks later is necessary to deem either one the best overall response.

TABLE 8
Response Assessment of Target and Non-Target Lesions

Target Lesions
Complete response (CR): Disappearance of all CNS target lesions sustained for at
least 4 weeks; with no new lesions
Partial response (PR); At least a 30% decrease in the sum longest diameter of CNS
target lesions, taking as reference the baseline sum longest diameter sustained for at
least four weeks; no new lesions
Progressive disease: At least a 20% increase in the sum longest diameter of CNS
target lesions, taking as reference the smallest sum on study (this includes the
baseline sum if that is the smallest on study). In addition to the relative increase of
20%, at least one lesion must increase by an absolute value of 5 mm or more to be
considered progression
Stable disease (SD); Neither sufficient shrinkage to qualify for partial response nor
sufficient increase to qualify for progressive disease, taking as reference the
smallest sum longest diameter while on study
Non-target Lesions
Complete response (CR): Requires all of the following: disappearance of all
enhancing CNS non-target lesions, no new CNS lesions
Non-complete response or non-progressive disease: Persistence of one or more non-
target CNS lesion or lesions
Progressive disease: Any of the following: unequivocal progression of existing
enhancing non-target CNS lesions, new lesion(s) (except while on immunotherapy-
based treatment), or unequivocal progression of existing tumor-related non-
enhancing (T2/FLAIR) CNS lesions. In the case of immunotherapy-based treatment,
new lesions alone may not constitute progressive disease

At each protocol-specified timepoint, a response assessment should occur and CNS assessments should be coincident with extra-CNS assessment. Table 9 shows the requirements to deem a partial response or complete response

TABLE 9
Summary of Response Criteria for CNS Metastases Proposed by mRECIST-BM

| | Complete | Partial | Stable | Progressive |
|---|---|---|---|---|
| | Response | Response | Disease | Disease |
| Target lesions | None | ≥30% decrease | <30% decrease | ≥20% increase |
| | | in sum longest | relative to | in sum longest |
| | | distance relative | baseline but <20% | distance relative |
| | | to baseline | increase in sum | to nadir* |
| | | | longest distance | |
| | | | relative to nadir | |
| Non-target | None | Stable or | Stable or | |
| lesions | | improved | improved | |
| New lesion(s) † | None | None | None | Present |
| Requirement | All | | | |
| for response | | | | |

*Progression occurs when this criterion is met.
† A new lesion is one that not present on prior scans and is visible in minimum two projections. If a new lesion is equivocal, for example because of its small size, continued therapy can be considered, and follow-up assessment will clarify if the new lesion is new disease. If repeat scans confirm there is definitely a new lesion, progression should be declared using the date of the initial scan showing the new lesion. For immunotherapy-based approaches, new lesions alone do not define progression.

### Assessment of Target and Non-Target CNS Lesions:

While on study, all CNS target lesions should have their actual measurement recorded, even if very small (e.g., 2 mm). If the lesion disappears, the value should be recorded as 0 mm. However, if the lesion is sufficiently small (but still present) to be assigned an exact measure, a default value of 5 mm should be recorded on the eCRF. Lesions might coalesce during treatment. As lesions coalesce, a plane between them may be maintained that would aid in obtaining maximum longest diameter of each individual lesion. If the lesions have truly coalesced such that they are no longer separable, the vector of the longest diameter in this instance should be the maximum longest diameter for the coalesced lesion. New lesions can appear during treatment. The finding of a new CNS lesion should be unequivocal and not due to technical or slice variation. A new lesion is one that was not present on prior scans. If the MRI is obtained with slice thickness of 1.5 mm or less, the new lesion should also be visible in axial, coronal, and sagittal reconstructions of 1.5 mm or thinner projections. If a new lesion is equivocal, for example because of its small size (i.e., ≤5 mm), continued therapy can be considered, and a follow-up assessment will clarify if it really is new disease. If repeated scans confirm a new lesion, progression should be declared using the date of the initial scan showing the new lesion. In the case of immunotherapy, however, new lesions alone cannot constitute progressive disease. Unequivocal progression of non-target lesions can merit discontinuation of therapy. When a patient also has measurable disease, to be deemed as having unequivocal progression on the basis of non-target disease alone there must also be an overall substantial worsening in non-target disease such that, even in the presence of stable disease or partial response in target disease, the overall tumor burden has increased sufficiently to merit discontinuation of therapy. When the patient has only non-measurable disease, there must be an overall level of substantial worsening to merit discontinuation of therapy. The mRECIST-BM group acknowledges the case of patients who have been treated with stereotactic radiosurgery or immunotherapy-based approaches, for whom there has been radiographical evidence of enlargement of target and non-target lesions, which do not necessarily represent tumor progression. If radiographical evidence of progression exists, but clinical evidence indicates that the radiological changes are due to treatment effect (and not to progression of cancer), additional evidence is needed to distinguish between true progression and treatment effect, in which case standard MRI alone is insufficient. The methods used to distinguish between true progression and treatment effect should be specified prospectively in the clinical protocol. Patients can be continued on protocol therapy pending further investigation with one or more of the following options. The scan can be repeated at the next protocol-scheduled assessment or sooner, and generally within about 6 weeks. An investigator can choose a shorter time interval if progressive symptoms or other clinical concerns arise. Continued tumor growth might be consistent with radiographical progression, in which case the patient should leave the study. Stabilization and shrinkage of a lesion can be consistent with treatment effect, in which case the patient can stay in the study. For patients with equivocal results even on the next restaging scan, the scan can be repeated again at a subsequent protocol-scheduled assessment or sooner, although surgery or use of an advanced imaging modality (in the case of stereotactic radiosurgery), or both, are strongly encouraged. Surgical pathology can be obtained via biopsy or resection.

For lesions treated by stereotactic radiosurgery, additional evidence of tumor progression or treatment effect (radionecrosis) can be acquired with an advanced imaging modality, such as perfusion MRI, magnetic resonance spectroscopy, or 18FLT or 18FDG PET. If subsequent testing shows that progression has occurred, the date of progression should be recorded as the date of the scan this issue was first raised. Patients can also have an equivocal finding on the scan (e.g., a small lesion that is not clearly new). Continued treatment is permissible until the next protocol-scheduled assessment. If the subsequent assessment shows that progression has indeed occurred, the date of progression should be recorded as the date of the initial scan where progression was suspected.

In patients receiving immunotherapy-based treatment, an initial increase in the number and size of metastases can be followed by radiographical stabilization or regression. This pattern might be related to the mechanism of action of immunotherapy, including immune infiltrates, and the time to mount an effective immune response. Thus, progressive disease should not be solely defined as by the appearance of new lesions but rather as a minimum 20% increase in the sum longest diameter of CNS target and new lesions, as unequivocal progression of existing enhancing non-target CNS lesions, as unequivocal progression of existing non-enhancing (T2/FLAIR) CNS lesions, or as clinical decline related to the tumor. If immune response-related radiographical changes are suspected, we advise to not change treatment until a short interval scan is obtained.

### Volumetric Criteria:

First, partial volumetric response should be defined as a 65% or greater decrease in the sum volume of CNS target lesions. Second, volumetric response should be reported as a waterfall plot to provide a global sense of potential efficacy. Third, in the absence of high quality data across multiple studies to show a clear correlation between lower volumetric thresholds and some measures of patient benefit, such as quality of life, neuro-cognitive function, or overall survival, it is premature to formally define a category of minor response or to lower the threshold at which to consider a volumetric response. However, we encourage digital archiving of trial images and accompanying linked clinical outcome data to allow for studies to be pooled to determine whether different cutpoints could be justified in the future.

### Treatment of Non-CNS (Extracranial) Disease:

Preclinical and clinical data sometimes show a differential response in intra-cranial versus extracranial locations, which could be related to inadequate drug penetration, differences in tumor microenvironment, or tumor heterogeneity between organ sites, among other possibilities. Many systemic agents are not expected to have CNS activity, primarily because of poor drug penetration. Local CNS therapies, such as whole-brain radiotherapy, stereotactic radiosurgery, or surgery, are not expected to affect extracranial sites at all. Traditionally, RECIST has used a summation of representative target lesions across all organ sites. Historically, patients with brain metastases have been excluded from systemic therapy trials. Even when included, patients with brain metastases often had to have stable, treated CNS lesions on study entry, and CNS lesions were rarely chosen as target lesions. The Macdonald and RANO-HGG criteria do not provide guidance about the treatment of extracranial disease, because extracranial disease is not relevant in most patients with primary brain tumors. The consequences have been an absence of flexibility to continue protocol therapy in the setting of discordant CNS versus non-CNS response or progression, a disincentive to image the brain as part of clinical trials, and the use of different definitions of response and progression endpoints in the local therapy trials and systemic therapy trials. We propose that CNS and non-CNS should be assessed as separate compartments (Table 10). As such, CNS response will be scored irrespectively of extracranial response and vice versa. For progression, CNS and non-CNS will be scored according to mRECIST-BM and RECIST 1.1 criteria, respectively (Table 11). If progression occurs in either or both compartments, the criteria for bi-compartmental progression-free survival will have been met. Protocols can also prospectively specify CNS progression-free survival and non-CNS progression-free survival as endpoints. Protocols should specify the plan for patients who progress in one compartment only. For example, a patient who develops isolated CNS progression in a systemic therapy trial can be given the option to have their CNS disease treated with whole brain radiotherapy, stereotactic radiosurgery, or surgery and remain on protocol therapy until the time of non-CNS disease progression, unacceptable toxicity, or death. The date of non-CNS progressive disease should be recorded when it occurs.

TABLE 10
CNS and Non-CNS Assessment

| CNS (mRECIST-BM) | Non-CNS (RECIST v1.1) | Response |
|---|---|---|
| Complete response, partial | Complete response, partial | Log as CNS and non-CNS |
| response, or stable disease | response, or stable disease | complete response, |
| | | partial response, |
| | | or stable diseases |
| Complete response, partial | Progressive disease | Log as CNS complete |
| response, or stable disease | | response, partial |
| | | response, or stable |
| | | disease; log as non-CNS |
| | | progressive disease |
| Progressive disease | Complete response, partial | Log as CNS progressive |
| | response, or stable disease | disease; log as non-CNS |
| | | complete response, |
| | | partial response, or |
| | | stable disease |
| Progressive disease | Progressive disease | Log as both CNS and non-CNS |
| | | progressive disease |

TABLE 11
Bi-compartmental Progression-Free Survival

| | Non-CNS | Bi-compartmental | |
|---|---|---|---|
| CNS (mRECIST-BM) | (RECIST v1 .1) | PFS | Note |
| Complete response | Progressive disease | Log as progression- | Log as non-CNS |
| partial response, | | free survival event | progressive disease |
| or stable disease | | | |
| Progressive disease | Complete response | Log as progression- | Log as CNS |
| | partial response, | free survival event | progressive disease |
| | or stable disease | | |
| Progressive disease | Progressive disease | Log as progression- | Log as both CNS |
| | | free survival event | and non-CNS |
| | | | progressive disease |

TABLE 12
OBJECTIVES AND ENDPOINTS

| | |
|---|---|
| Objectives | Endpoints |

Primary

| | |
|---|---|
| To determine the maximum tolerated | Occurrence of Dose-limiting |
| dose (MTD) and/or the recommended | toxicities (DLTs). |
| Phase 2 dose (RP2D) for Compound | Nature and frequency of AEs, |
| Ia alone and in combination with | pharmacodynamic (PD) and |
| cobimetinib in one or more schedules. | Compound Ia pharmacokinetic |
| | (PK) profile and clinical activity |
| | guiding selection of RP2D. |

Secondary

| | |
|---|---|
| To evaluate the safety and tolerability | Frequency, nature, and severity of |
| of Compound Ia alone and in | adverse effects (AEs), with |
| combination with cobimetinib. | severity determined according to |
| | the National Cancer Institute |
| | Common Terminology Criteria for |
| | Adverse Events (NCI CTCAE) |
| | v5.0. |
| | Change from baseline in vital |
| | signs, physical and neurological |
| | findings, ECG readings and left |
| | ventricular ejection fraction |
| | (LVEF) parameters or clinical |
| | laboratory test results. |
| To evaluate the clinical activity of | Objective response rate (ORR), |
| Compound Ia alone and in | disease control rate (DCR), |
| combination with cobimetinib | duration of response according to |
| | Response Evaluation Criteria in |
| | Solid Tumors (RECIST) v1.1 |
| | (extracranial disease) and modified |
| | response assessment in neuro- |
| | oncology for brain metastases |
| | (mRECIST-BM; intra-cranial |
| | disease) criteria. |
| PK of Compound Ia (and cobimetinib, | PK parameters such as maximum |
| if applicable) in plasma following | concentration (Cₘₐₓ), time of |
| oral administration of Compound Ia | maximum concentration observed |
| administered alone and in | (Tₘₐₓ), area under the curve |
| combination with cobimetinib. | (AUC), and half-life (T_{1/2}) for |
| | Compound Ia and cobimetinib (if |
| | applicable). |
| To determine the effect of food on PK | Effect of a meal on PK parameters |
| of Compound Ia. | such as Cₘₐₓ, Tₘₐₓ, AUC, and T_{1/2} |
| | for Compound Ia following oral |
| | administration of Compound Ia |
| | compared to fasting conditions. |

Exploratory

| | |
|---|---|
| To assess the PD effects of | Imaging parameters such as |
| Compound Ia by changes in | standardized uptake value (SUV) |
| fluorodeoxyglucose positron emission | and number of metabolic |
| tomography (FDG-PET) to guide | responses. |
| dose selection. | |
| To confirm BRAF-V600 status | Detection of BRAF-V600 mutation |
| retrospectively and assess | in archival tumor biopsy and |
| concordance between tumor DNA and | blood. |
| plasma circulating tumour DNA | Comparison of the mutation |
| (ctDNA). | prevalence at baseline between |
| | tumor DNA (next-generation |
| | sequencing [NGS]; NGS-based |
| | FoundationOne CDx) and plasma |
| | ctDNA (NGS-based |
| | FoundationOne Liquid CDx). |
| To measure PD of Compound Ia | Change from baseline in the |
| on molecular targets in blood and | fraction of ctDNA (mean tumor |
| cerebrospinal fluid (CSF) (where | molecules [MTM/mL]) and in |
| applicable) and guide dose | the ctDNA status (positive or |
| selection in one or more schedules | negative) over time, assessed by |
| (where applicable). | massively multiplexed PCR and |
| | NGS (mmPCR-NGS). |
| To evaluate the relationship | Relationship between plasma |
| between drug exposure in plasma | and CSF exposure (where |
| and CSF (where applicable) and | applicable) or PK parameters |
| safety, PD, and/or clinical activity | (such as Cₘₐₓ, AUC) for |
| of Compound Ia. | Compound Ia alone and in |
| | combination with cobimetinib |
| | for safety, PD, and clinical |
| | efficacy endpoints. |
| To evaluate Compound Ia CNS | CSF concentrations of |
| penetration. | Compound Ia (where |
| | applicable). |
| To investigate the presence of | To assess relative abundance |
| Compound Ia-derived | and PK parameters of any |
| metabolite(s) in plasma. | metabolite as appropriate. |
| To evaluate the endogenous | Plasma concentrations and |
| biomarkers | percentage change from baseline |
| 4β-hydroxycholesterol (4βHC), | of 4βHC, 4αHC and TC. |
| 4α-hydroxycholesterol (4 αHC) | |
| and total cholesterol (TC) for the | |
| assessment of CYP3A activity | |
| following oral administration of | |
| Compound Ia. | |

TABLE 13
Sampling schedule: Hourly Sample Collection Details

| | | | | | | Blood | | | | | |
|---|---|---|---|---|---|---|---|---|---|---|---|
| | | | | | | for | | | | | |
| | | | | Comp. | cobimetinib | endogenous | | | Archiv. | | |
| | | | | Ia PK | PK for Part | CYP | | | tumour | CSF | |
| | | Sched. | | QD/ | 1b and Part 2 | activation | Blood | Blood | tissue | for | |
| Cycle (28 | | time (h) | Time | BID/ | if applicable | markers | for | for | for | Comp. | CSF for |
| days) | Day | (a) | window | TID | (b) | (b) | BRAF | ctDNA | BRAF | Ia PK | ctDNA |
| Screening / | D-28 | | | | | x | | | x | | |
| Baseline | to | | | | | | | | | | |
| | D-1 | | | | | | | | | | |
| Cycle 1 | Day | Predose | | x | x | x | x | x | | | |
| | 1 | 0.5 | ±5 | x | x | | | | | | |
| | | | min | | | | | | | | |
| | | 1 | ±10 | x | x | | | | | | |
| | | | min | | | | | | | | |
| | | 2 | ±15 | x | x | | | | | | |
| | | | min | | | | | | | | |
| | | 4 | ±15 | x | x | | | | | | |
| | | | min | | | | | | | | |
| | | 5 | ±15 | x | x | | | | | | |
| | | | min | | | | | | | | |
| | | 6 | ±30 | x | x | | | | | | |
| | | | min | | | | | | | | |
| | | 8 (c, d) | ±30 | x | x | | | | | | |
| | | | min | | | | | | | | |
| | Day | Predose | | x | x | x | | | | | |
| | 2 | (d) | | | | | | | | | |
| | Day | Predose | | x | x | x | | | | | |
| | 8 | 1 (d) | ±15 | x | x | | | | | x | x |
| | | | min | | | | | | | | |
| | Day | Predose | | x | x | x | | x | | | |
| | 15 | 0.5 | ±5 | x | x | | | | | | |
| | | | min | | | | | | | | |
| | | 1 | ±10 | x | x | | | | | | |
| | | | min | | | | | | | | |
| | | 2 | ±15 | x | x | | | | | | |
| | | | min | | | | | | | | |
| | | 4 | ±15 | x | x | | | | | | |
| | | | min | | | | | | | | |
| | | 5 | ±15 | x | x | | | | | | |
| | | | min | | | | | | | | |
| | | 6 | ±30 | x | x | | | | | | |
| | | | min | | | | | | | | |
| | | 8 (c,d) | ±30 | x | x | | | | | | |
| | | | min | | | | | | | | |
| | Day | Predose | | x | x | x | | | | | |
| | 16 | (d) | | | | | | | | | |
| | Day | Predose | | x | x | x | | | | | |
| | 22 | 1 (d) | ±15 | x | x | | | | | | |
| | | | min | | | | | | | | |
| Cycle 2 | | Predose | | x | x | x | | x | | | |
| | Day | 1 (d) | ±15 | x | x | | | | | x (i) | x (i) |
| | 1 | | min | | | | | | | | |
| | Day | Predose | | x | x | x | | | | | |
| | 15 | (d) | | | | | | | | | |
| Cycle 3 | Day | Predose | | x | x | x | | x | | | |
| | 1 | | | | | | | | | | |
| Cycle 5 | Day | Predose | | x (to | x (to be | x (to be | | x (to be | | | |
| | 1 | | | be | collected for | collected | | collected | | | |
| | | | | collected | Part 2 only) | for | | for Part | | | |
| | | | | for | | Part 2 | | 2 only) | | | |
| | | | | Part 2 | | only) | | | | | |
| | | | | only) | | | | | | | |
| Cycle 7 | Day | Predose | | x (to | x (to be | x (to be | | x (to be | | | |
| | 1 | | | be | collected for | collected | | collected | | | |
| | | | | collected | Part 2 only) | for | | for Part | | | |
| | | | | for | | Part 2 | | 2 only) | | | |
| | | | | Part 2 | | only) | | | | | |
| | | | | only) | | | | | | | |
| Discontinuation | | | | x | x | x | x (to | X | | | |
| visit | | | | | | | be | | | | |
| (e) | | | | | | | collected | | | | |
| | | | | | | | for | | | | |
| | | | | | | | Part 2 | | | | |
| | | | | | | | only) | | | | |
| Unscheduled | | | | x | x | x | | x | | x | x |
| visit in | | | | | | | | | | | |
| case of | | | | | | | | | | | |
| missed | | | | | | | | | | | |
| samples | | | | | | | | | | | |
| Unscheduled | | | | x | x | x | | | | | |
| visit in | | | | | | | | | | | |
| case of | | | | | | | | | | | |
| DLT, ≥ | | | | | | | | | | | |
| Grade 3 AE | | | | | | | | | | | |
| leading to | | | | | | | | | | | |
| dose | | | | | | | | | | | |
| interruption, | | | | | | | | | | | |
| clinical | | | | | | | | | | | |
| significant | | | | | | | | | | | |
| ECG/ECHO/ | | | | | | | | | | | |
| MUGA | | | | | | | | | | | |

(a) “Predose” is a sample collection to be conducted shortly before the time of drug administration (“Dosing”) of Compound Ia and cobimetinib (if applicable). Both Compound Ia and cobimetinib (if applicable) will be taken at the same time in the morning. Scheduled time points refer to times in hours after drug dosing of Compound Ia and cobimetinib (if applicable). Time of PK sample collection needs to be recorded and entered in the eCRF for Compound Ia and cobimetinib (if applicable).
(b) In case of no drug-drug interaction between Compound Ia and cobimetinib, cobimetinib PK samples and/or endogenous CYP markers samples might not be further collected in the Food Cohort or Part 2. In case Part 2 consists of treatment with Compound Ia alone, no cobimetinib PK samples will be taken.
(c) In case of a short half-life of Compound Ia assessed in the Pilot PK Cohort, the samples 8 hours after dosing at C1D1 and C1D15, and the C1D2, C1D3, C1D4 and C1D16 samples might not be further collected in the main dose-escalation, and/or Food Cohort and/or expansion cohorts (Part 2).
(d) In case of sufficient data for PK characterization of Compound Ia during study conduct, these samples might not be further collected in Part 2.
(e) If the Discontinuation Visit coincides with an Unscheduled Visit, all planned assessments for the Discontinuation Visit need to be performed.

### DETAILED DESCRIPTION OF FIGURES

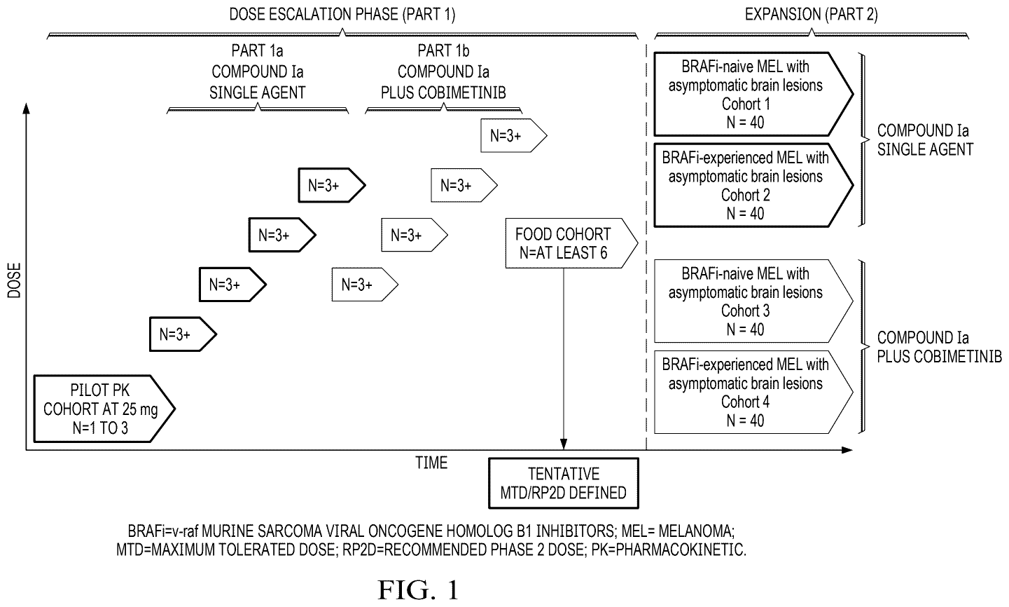

FIG. 1 Overview of phase Ia/b study design: Part 1 is a dose escalation phase. In a Cohort of part 1a Compound Ia is used in monotherapy, and in a Cohort of part 1b the Compound Ia is used in combination with cobimetinib. In addition, part I comprises a Pilot PK Cohort and a Food Cohort. Part 2 is an expansion phase with four cohorts: BRAFi-naive melanoma with asymptomatic brain lesions corresponds to Cohort 1 (Compound Ia monotherapy) and Cohort 3 (Compound Ia plus cobimetinib). BRAFi-experienced melanoma with asymptomatic brain lesions corresponds to Cohort 2 (Compound Ia monotherapy) and Cohort 4 (Compound Ia plus cobimetinib).

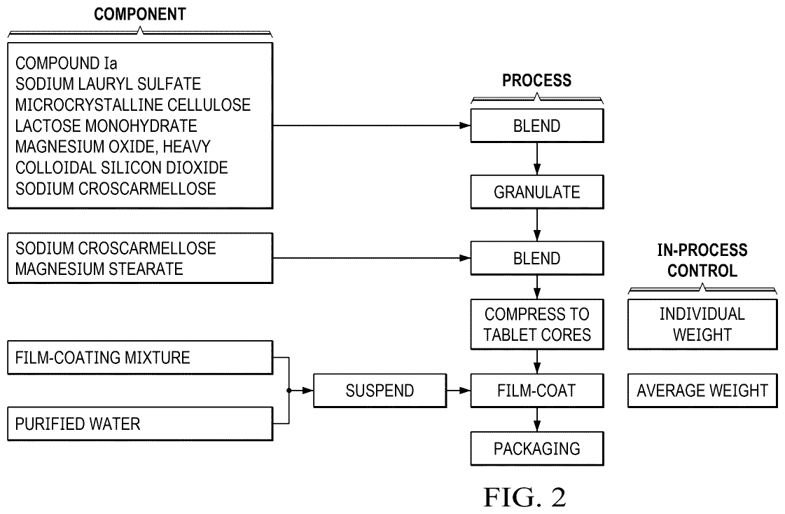

FIG. 2 Manufacturing process flow chart for a film-coated tablet (Example A) comprising Compound Ia. A dry granulation process is used to manufacture the drug product:
Step 1: Weigh Compound Ia, sodium lauryl sulfate, microcrystalline cellulose, lactose monohydrate, magnesium oxide, colloidal silicon dioxide and sodium croscarmellose, and blend. Spray drying of Compound Ia is optionally performed as Step 0.
Step 2: Dry granulation of the blend obtained from step 1. Alternatively, instead of dry granulation, high shear mixing or fluid bed granulation can be used on the blend obtained from step 1.
Step 3: Weigh sodium croscarmellose and magnesium stearate. Add them to the granulate obtained from step 2 and blend.
Step 4: Compress the blend obtained from step 3 to tablets and check the individual weight of tablet cores (as in-process control).
Step 5: Weigh the film-coating mixture and suspend it in purified water. Spray the obtained coating suspension onto tablet cores from step 4. The average weight of film coated tablets is measured (as in-process control).
Step 6: Film coated tablets are packaged and labeled for clinical use.

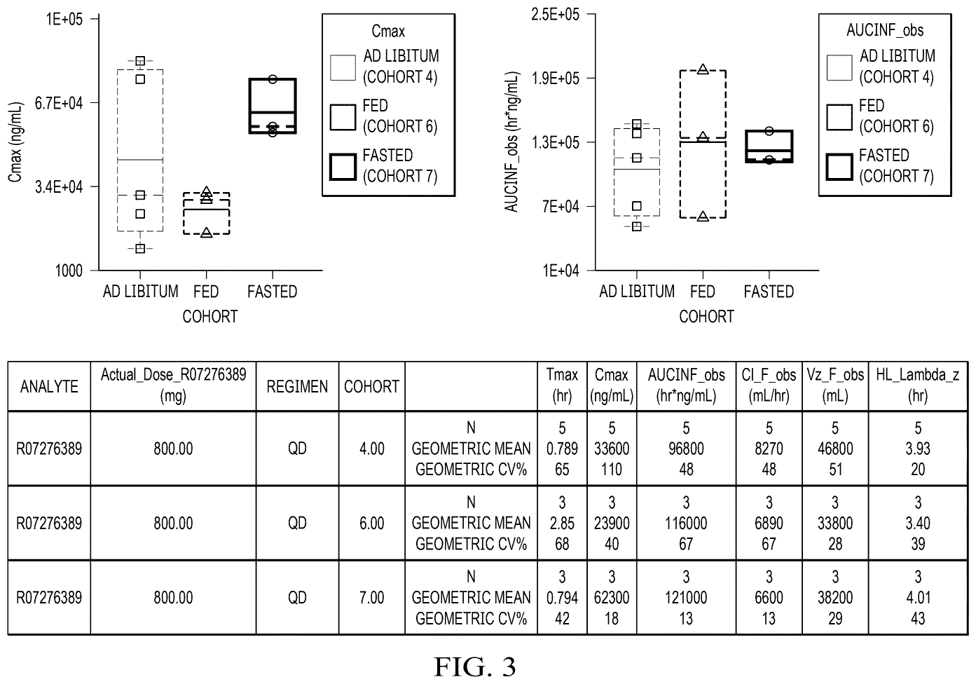

FIG. 3: Preliminary PK data for regarding the effect of food indicates that the intake of food resulted in lower Cₘₐₓ for the fed cohort, but surprisingly did not significantly alter the AUCINF (AUC_{0-∞}) for the Compound Ia (also known as RO7276389).

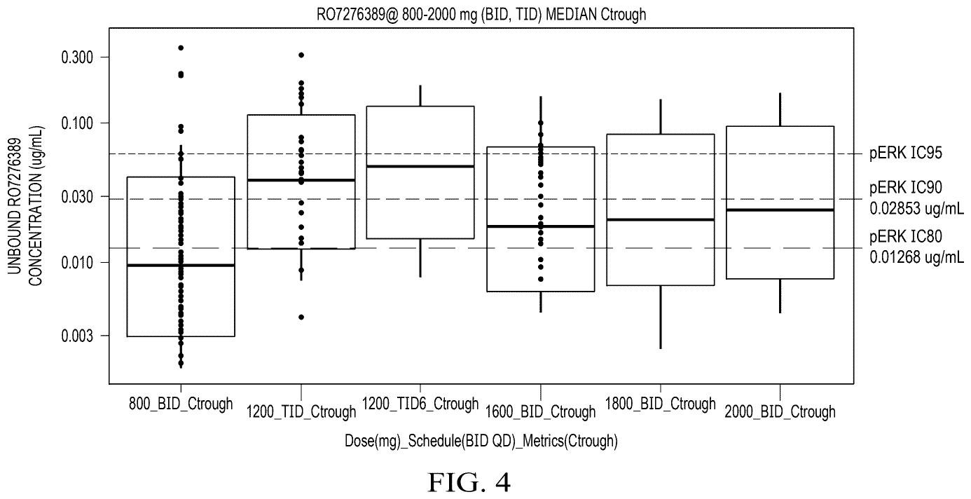

FIG. 4: A trough concentration (C_{trough}) simulation for Compound Ia (also known as RO7276389) based on a Preliminary Population Pharmacokinetic (PopPK) Model suggests coverage around IC95 for 1200 mg TID and 1600 mg BID. Such coverage is substantially higher than the C_{trough} coverage of currently available BRAF inhibitors at the approved dose, which result in coverage around IC80 and are limited by dose-limiting toxicities. Importantly, this simulation also suggests that the highest coverage can be obtained with a dosage regime herein termed as 1200 mg TID6, wherein the first daily dose is administered in the morning with the first meal, the second daily dose is then administered 6 hours later (possibly with a second meal), and the third daily dose is administered another 6 hours later (possibly with a third meal).

### Pharmaceutical Compositions:

One embodiment of the invention provides a pharmaceutical composition for use according to the invention, wherein the composition contains the compound of formula (I) and one or more therapeutically inert carriers, diluents or excipients, as well as a method to prepare such a pharmaceutical compositions. In one example, the compound of formula (I) or a pharmaceutically acceptable salt thereof, may be formulated by mixing at ambient temperature at the appropriate pH, and at the desired degree of purity, with physiologically acceptable carriers, i.e., carriers that are non-toxic to recipients at the dosages and concentrations employed into a galenical administration form. The compound of formula (I) exhibits a low and pH dependent solubility. It behaves like a weak acid across the physiological pH range that is poorly soluble at low pH with increasing solubility at neutral and alkaline pH. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

As used herein, a “pharmaceutically acceptable carrier” or “pharmaceutically acceptable excipient” is intended to include any and all material compatible with pharmaceutical administration including solvents, dispersion media, coatings, enteric coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Pharmaceutical compositions can be obtained by processing the compound of formula (I) with pharmaceutically acceptable, inorganic or organic carriers or excipients. Lactose, corn starch or derivatives thereof, talc, stearic acids or it's salts and the like can be used, for example, as such carriers for tablets, film-coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain fillers, pH-modifiers, glidants, disintegrants, lubricants, preservatives, solubilizers, stabilizers, enteric coatings, wetting agents, emulsifiers, sweeteners, colorants, flavorants, salts for varying the osmotic pressure, buffers, masking agents or antioxidants. They can also contain still other therapeutically valuable substances. Suitable fillers for tablets are, for example, microcrystalline cellulose, lactose monohydrate, anhydrous lactose, mannitol, isomalt and dibasic dicalcium phosphate. Suitable wetting agents for tablets are, for example, Sodium Lauryl Sulfate, Polyvinylpyrrolidone/Polyvinyl alcohol (PVP/PVA) copolymer and hypromellose. Suitable pH-modifier for tablets are, for example, magnesium oxide, calcium carbonate, calcium hydrogen carbonate, lysine and tromethamine. A suitable glidants for tablets is, for example, colloidal silicon dioxide. Suitable lubricants for tablets are, for example magnesium stearate, sodium stearyl fumarate and macrogol 6000.

Pharmaceutical compositions of the Compound Ia can be prepared for storage by mixing the active ingredient having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions.

Pharmaceutical compositions of the compound of formula (I) include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of the compound of formula (I) which produces a therapeutic effect. Generally, out of one hundred percent, this amount will range from about 1 percent to about 90 percent of active ingredient, preferably from about 2 percent to about 70 percent, most preferably from about 4 percent to about 40 percent. Methods of preparing these compositions include the step of bringing into association the compound of formula (I) with the carrier and, optionally, one or more accessory ingredients. In general, the pharmaceutical compositions can be prepared by uniformly and intimately bringing into association the compound of formula (I) with liquid carriers, or finely divided solid carriers, or both, and then, if necessary, shaping the product. Pharmaceutical compositions suitable for oral administration may be in the form of capsules, cachets, sachets, pills, tablets, film-coated tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the compound of formula (I) as an active ingredient. The compound of formula (I) may also be administered as a bolus, electuary or paste.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

The formulations to be used for in vivo administration must be sterile. This can be readily accomplished by filtration through sterile filtration membranes.

The dosage can vary within wide limits and will, of course, have to be adjusted to the individual requirements in each particular case. In the case of oral administration the dosage for adults can vary from about 100 mg to about 4000 mg per day, preferably about 1000 mg to about 4000 mg per day, more preferably about 1600 mg to about 4000 mg per day, of a compound of general formula (I) or of the corresponding amount of a pharmaceutically acceptable salt or solvate thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical composition conveniently contains about 5 mg to about 800 mg, particularly about 10 mg to about 700 mg, more particularly about 25 mg to about 600 mg of a compound of formula (I) or a pharmaceutically acceptable salt thereof. In one embodiment the pharmaceutical composition contains about 50 mg to about 200 mg of a compound of formula (I) or a pharmaceutically acceptable salt thereof. In one embodiment the pharmaceutical composition contains about 200 mg to about 400 mg of a compound of formula (I) or a pharmaceutically acceptable salt thereof. In one embodiment the pharmaceutical composition contains about 400 mg to about 600 mg of a compound of formula (I) or a pharmaceutically acceptable salt thereof. In one embodiment the pharmaceutical composition contains about 600 mg to about 800 mg of a compound of formula (I) or a pharmaceutically acceptable salt thereof. In one embodiment the pharmaceutical composition contains about 800 mg to about 1000 mg of a compound of formula (I) or a pharmaceutically acceptable salt thereof. In one embodiment the pharmaceutical compositions contains about 25 mg of a compound of formula (I) or a pharmaceutically acceptable salt thereof. In one embodiment the pharmaceutical composition contains about 200 mg of a compound of formula (I) or a pharmaceutically acceptable salt thereof. In a certain embodiment a pharmaceutical composition comprising a compound of formula (I) contains in addition about 1-500 mg, in particular 5-100 mg, more particularly 60 mg, of a MEK inhibitor, in particular cobimetinib, in a fixed-dose combination. In a certain embodiment a pharmaceutical composition comprising a compound of formula (I) contains in addition about 20 mg, of a MEK inhibitor, in particular cobimetinib, in a fixed-dose combination.

Non-limiting examples of pharmaceutical compositions according to the invention are:

### Example A—Film-Coated Tablet

Film-coated tablets can be manufactured by conventional dry granulation followed by tablet compression and film coating. Non-limiting examples of film-coated tablets according to the invention can be of the following composition:

TABLE 14
possible film-coated tablet compositions;

| Component | Function | mg/tablet | |
|---|---|---|---|
| Compound Ia | Active | 25.000 | 200.000 |
| | ingredient | | |
| Sodium Lauryl Sulfate | Wetting agent | 3.125 | 4.000 |
| Microcrystalline Cellulose | Filler | 368.750 | 305.000 |
| Lactose monohydrate | Filler | 187.500 | 155.000 |
| Magnesium Oxide | pH-modifier | 12.500 | 100.000 |
| Colloidal silicon dioxide | Glidant | 3.125 | 4.000 |
| Sodium Croscarmellose | Disintegrant | 18.750 | 24.000 |
| Magnesium Stearate | Lubricant | 6.250 | 8.000 |
| Subtotal Weight (tablet core) | | 625.000 | 800.000 |
| Film Coat | | | |
| Opadry II 85F240172 PINK¹ | | 18.750 | 24.000 |
| Purified Water² | | qs | qs |
| Total Weight (film coated | | 643.750 | 824.000 |
| tablet) | | | |

¹The film-coating mixture consists of compendial components: polyvinyl alcohol, partially hydrolyzed, macrogol 3350, Talc, titanium dioxide, iron oxide yellow, and iron oxide red.
²Purified water is used for aqueous film coating; it is essentially removed during processing; qs: quantum satis.

### Manufacturing Procedure of the Film-Coated Tablets

A dry granulation process can be used to manufacture the drug product:
Step 1: Weigh Compound Ia, sodium lauryl sulfate, microcrystalline cellulose, lactose monohydrate, magnesium oxide, colloidal silicon dioxide and sodium croscarmellose, and blend. Spray drying of Compound Ia is optionally performed as Step 0.
Step 2: Dry granulation of the blend obtained from step 1. Alternatively, instead of dry granulation, high shear mixing or fluid bed granulation can be used on the blend obtained from step 1.
Step 3: Weigh sodium croscarmellose and magnesium stearate. Add them to the granulate obtained from step 2 and blend.
Step 4: Compress the blend obtained from step 3 to tablets and check the individual weight of tablet cores (as in-process control).
Step 5: Weigh the film-coating mixture and suspend it in purified water. Spray the obtained coating suspension onto tablet cores from step 4. The average weight of film coated tablets is measured (as in-process control).
Step 6: Film coated tablets are packaged and labeled.

The Components of the tablet core are:
The formulation comprises a filler or a combination of at least two fillers that ensure the desired mechanical resistance of the tablet. The desired mechanical resistance is characterized by sufficient tensile strengths, low friability and low abrasion of tablet cores so that they withstand stresses during coating and packaging operations. These fillers can be for instance microcrystalline cellulose and lactose monohydrate in the ratio of about 2:1.
The formulation contains a wetting agent, such as for instance sodium lauryl sulfate (0.5% by weight relative to the total weight of the uncoated tablet).
The formulation contains a glidant, such as for instance colloidal silicon dioxide (0.5% by weight relative to the total weight of the uncoated tablet).
The formulation contains an alkaline pH-modifier, such as for instance magnesium oxide, in a ratio of 1:2 to Compound Ia. The pH modifier is intended to create a favorable in vivo micro-pH environment for the solubility of the API (active pharmaceutical ingredient).
The formulation contains a disintegrant, such as for instance sodium croscarmellose (3.0% by weight relative to the total weight of the uncoated tablet). Half is added prior, half after granulation.
The formulations contains magnesium stearate as lubricant (1.0% by weight relative to the total weight of the uncoated tablet).

The components of the film-coating are:
A film-coating mixture containing polyvinyl alcohol as film former, macrogol 3350 as softener, talc as anti-tackling agent, titanium dioxide and, red and yellow iron oxide as pigments.
The film-coating mixture is dispersed in purified water at a solid content of 15%. The film coat is sprayed onto the tablet cores in a pan coater aiming at a weight gain of 3% relative to the weight of the tablet cores.

Description of the film-coated tablet: The Drug Product is a pink, oblong film-coated tablet. The 25 mg dose strength contains 4% of Compound Ia. The 200 mg film-coated tablets contain 25% of Compound Ia.

### Example B—Film-Coated Tablet

Film-coated tablets can be manufactured by conventional dry granulation followed by tablet compression and film coating. Non-limiting examples of film-coated tablets according to the invention can be of the following composition:

TABLE 15
possible film-coated tablet compositions;

| Component | Function | mg/tablet | |
|---|---|---|---|
| Compound Ia | Active | 50.000 | 300.000 |
| | ingredient | | |
| Hypromellose or PVP/PVA | Wetting agent | 1.250 | 8.000 |
| copolymer | | | |
| Microcrystalline Cellulose | Filler | 361.667 | 221.333 |
| Lactose monohydrate | Filler | 180.833 | 110.667 |
| Magnesium Oxide | pH-modifier | 12.500 | 100.000 |
| Colloidal silicon dioxide | Glidant | 3.125 | 4.000 |
| Crospovidone | Disintegrant | 12.500 | 40.000 |
| Sodium stearyl fumarate | Lubricant | 3.125 | 16.000 |
| Subtotal Weight (tablet core) | | 625.000 | 800.000 |
| Film Coat | | | |
| Opadry II 85F240172 PINK¹ | | 18.750 | 24.000 |
| Purified Water² | | qs | qs |
| Total Weight (film coated | | 643.750 | 824.000 |
| tablet) | | | |

¹The film-coating mixture consists of compendial components: polyvinyl alcohol, partially hydrolyzed, macrogol 3350, Talc, titanium dioxide, iron oxide yellow, and iron oxide red.
²Purified water is used for aqueous film coating; it is essentially removed during processing; qs: quantum satis.

### Manufacturing Procedure of the Film-Coated Tablets

A dry granulation process is used to manufacture the drug product:
Step 1: Weigh Compound Ia, Hypromellose or PVP/PVA copolymer, microcrystalline cellulose, lactose monohydrate, magnesium oxide, colloidal silicon dioxide and crospovidone and blend.
Step 2: Dry granulation of the blend obtained from step 1. Alternatively, instead of dry granulation, high shear mixing or fluid bed granulation can be used on the blend obtained from step 1.
Step 3: Weigh sodium crospovidone and sodium stearyl fumarate. Add them to the granulate obtained from step 2 and blend.
Step 4: Compress the blend obtained from step 3 to tablets and check the individual weight of tablet cores (as in-process control).
Step 5: Weigh the film-coating mixture and suspend it in purified water. Spray the obtained coating suspension onto tablet cores from step 4. Spray drying is optionally performed. The average weight of film coated tablets is measured (as in-process control).
Step 6: Film coated tablets are packaged and labeled.

### Example C—Film-Coated Tablet

Film-coated tablets can be manufactured by conventional dry granulation followed by tablet compression and film coating. Non-limiting examples of film-coated tablets according to the invention can be of the following composition:

TABLE 16
possible film-coated tablet compositions;

| Component | Function | mg/tablet | |
|---|---|---|---|
| Compound Ia | Active | 25.000 | 200.000 |
| | ingredient | | |
| Sodium Lauryl Sulfate | Wetting agent | 3.125 | 4.000 |
| Microcrystalline Cellulose | Filler | 368.750 | 305.000 |
| Lactose monohydrate | Filler | 187.500 | 155.000 |
| Calcium carbonate | pH-modifier | 12.500 | 100.000 |
| Colloidal silicon dioxide | Glidant | 3.125 | 4.000 |
| Sodium Croscarmellose | Disintegrant | 18.750 | 24.000 |
| Magnesium Stearate | Lubricant | 6.250 | 8.000 |
| Subtotal Weight (tablet core) | | 625.000 | 800.000 |
| Film Coat | | | |
| Opadry II 85F240172 PINK¹ | | 18.750 | 24.000 |
| Purified Water² | | qs | qs |
| Total Weight (film coated | | 643.750 | 824.000 |
| tablet) | | | |

¹The film-coating mixture consists of compendial components: polyvinyl alcohol, partially hydrolyzed, macrogol 3350, Talc, titanium dioxide, iron oxide yellow, and iron oxide red.
²Purified water is used for aqueous film coating; it is essentially removed during processing; qs: quantum satis.

### Manufacturing Procedure of the Film-Coated Tablets

A dry granulation process is used to manufacture the drug product:
Step 1: Weigh Compound Ia, sodium lauryl sulfate, microcrystalline cellulose, lactose monohydrate, calcium carbonate, colloidal silicon dioxide and sodium croscarmellose, and blend. Spray drying of Compound Ia is optionally performed as Step 0.
Step 2: Dry granulation of the blend obtained from step 1. Alternatively, instead of dry granulation, high shear mixing or fluid bed granulation can be used on the blend obtained from step 1.
Step 3: Weigh sodium croscarmellose and magnesium stearate. Add them to the granulate obtained from step 2 and blend.
Step 4: Compress the blend obtained from step 3 to tablets and check the individual weight of tablet cores (as in-process control).
Step 5: Weigh the film-coating mixture and suspend it in purified water. Spray the obtained coating suspension onto tablet cores from step 4. The average weight of film coated tablets is measured (as in-process control).
Step 6: Film coated tablets are packaged and labeled,

TABLE 17
Equipment used in the manufacturing
process of Examples A, B and C

| Process | | | |
|---|---|---|---|
| Step | Class | Subclass | Equipment |
| Blending | Diffusion | Bin Blender | e.g. Turbula |
| | mixer | | Blender, Servolift |
| | | | Blender |
| Granulation | Dry Granulator | Roller | e.g. Gerteis |
| | | Compaction | Minipactor |
| Tablet | Tablet Press | Power Assisted | e.g. Fette 1090i or |
| Compression | | | Korsch XL100 |
| Film-Coating | Pan Coating | Perforated | e.g. Glatt GC750 |
| | | Coating System | |

The invention further relates to:
A certain embodiment of the present invention relates to the compound of formula (I), or a pharmaceutically acceptable salt thereof, alone or in combination with cobimetinib, for use in the treatment of BRAF-mutant metastatic or locally advanced solid tumours;
A certain embodiment of the present invention relates to the compound of formula (I), or a pharmaceutically acceptable salt thereof, alone or in combination with cobimetinib, for use in the treatment of BRAF-mutant metastatic melanoma with brain metastases;
A certain embodiment of the present invention relates to a film-coated tablet comprising the compound of formula (I) or a pharmaceutically acceptable salt thereof, wherein the tablet composition further comprises a pH-modifier and thereby enhances the dissolution properties of Compound Ia;
A certain embodiment of the present invention relates to the compound of formula (I), or a pharmaceutically acceptable salt thereof, alone or in combination with cobimetinib, for use in the treatment of BRAF-mutant metastatic melanoma with brain metastases, wherein the melanoma was treated with a checkpoint inhibitor;
A certain embodiment of the present invention relates to the compound of formula (I), or a pharmaceutically acceptable salt thereof, in combination with cobimetinib, for use in the treatment of BRAF-mutant metastatic melanoma with brain metastases, wherein the melanoma was treated with a checkpoint inhibitor;
A certain embodiment of the present invention relates to the compound of formula (I), or a pharmaceutically acceptable salt thereof, for use in the treatment of BRAF-mutant metastatic melanoma with brain metastases, wherein the melanoma was treated with a checkpoint inhibitor;
A certain embodiment of the present invention relates to the compound of formula (I), or a pharmaceutically acceptable salt thereof, alone or in combination with cobimetinib, for use in the treatment of BRAF-mutant metastatic melanoma with brain metastases, wherein the melanoma was previously treated with a checkpoint inhibitor;
A certain embodiment of the present invention relates to the compound of formula (I), or a pharmaceutically acceptable salt thereof, in combination with cobimetinib, for use in the treatment of BRAF-mutant metastatic melanoma with brain metastases, wherein the melanoma was previously treated with a checkpoint inhibitor;
A certain embodiment of the present invention relates to the compound of formula (I), or a pharmaceutically acceptable salt thereof, for use in the treatment of BRAF-mutant metastatic melanoma with brain metastases, wherein the melanoma was previously treated with a checkpoint inhibitor;
A certain embodiment of the present invention relates to the compound of formula (I), or a pharmaceutically acceptable salt thereof, alone or in combination with cobimetinib, for use in the treatment of BRAF-mutant metastatic melanoma with brain metastases, wherein the melanoma was previously treated with a checkpoint inhibitor and a BRAF inhibitor;
A certain embodiment of the present invention relates to the compound of formula (I), or a pharmaceutically acceptable salt thereof, in combination with cobimetinib, for use in the treatment of BRAF-mutant metastatic melanoma with brain metastases, wherein the melanoma was previously treated with a checkpoint inhibitor and a BRAF inhibitor;
A certain embodiment of the present invention relates to the compound of formula (I), or a pharmaceutically acceptable salt thereof, for use in the treatment of BRAF-mutant metastatic melanoma with brain metastases, wherein the melanoma was previously treated with a checkpoint inhibitor and a BRAF inhibitor;
A certain embodiment of the present invention relates to the compound of formula (I), or a pharmaceutically acceptable salt thereof, alone or in combination with cobimetinib, for use in the treatment of BRAF-mutant metastatic or locally advanced solid tumours, wherein the tumour was previously treated with a checkpoint inhibitor;
A certain embodiment of the present invention relates to the compound of formula (I), or a pharmaceutically acceptable salt thereof, in combination with cobimetinib, for use in the treatment of BRAF-mutant metastatic or locally advanced solid tumours, wherein the tumour was previously treated with a checkpoint inhibitor;
A certain embodiment of the present invention relates to the compound of formula (I), or a pharmaceutically acceptable salt thereof, for use in the treatment of BRAF-mutant metastatic or locally advanced solid tumours, wherein the tumour was previously treated with a checkpoint inhibitor;
A certain embodiment of the present invention relates to the compound of formula (I), or a pharmaceutically acceptable salt thereof, alone or in combination with cobimetinib, for use in the treatment of BRAF-mutant metastatic or locally advanced solid tumours, wherein the tumour was previously treated with a checkpoint inhibitor and a BRAF inhibitor;
A certain embodiment of the present invention relates to the compound of formula (I), or a pharmaceutically acceptable salt thereof, for use in the treatment of BRAF-mutant metastatic or locally advanced solid tumours, wherein the compound of formula (I) is administered orally with or without food;
A certain embodiment of the present invention relates to the compound of formula (I), or a pharmaceutically acceptable salt thereof, for use in the treatment of BRAF-mutant metastatic or locally advanced solid tumours, wherein the compound of formula (I) is administered orally with water;
A certain embodiment of the present invention relates to the compound of formula (I), or a pharmaceutically acceptable salt thereof, for use in the treatment of BRAF-mutant metastatic or locally advanced solid tumours, wherein the compound of formula (I) is administered orally with food, in particular after a high-fat meal;
A certain embodiment of the present invention relates to the compound of formula (I), or a pharmaceutically acceptable salt thereof, for use in the treatment of BRAF-mutant metastatic or locally advanced solid tumours, wherein the compound of formula (I) is administered orally without food, in particular following a 10 hour fast;
A certain embodiment of the present invention relates to the compound of formula (I), or a pharmaceutically acceptable salt thereof, for use in the treatment of BRAF-mutant metastatic or locally advanced solid tumours, wherein the compound of formula (I) or a pharmaceutically acceptable salt thereof is administered orally three times a day with about 6 hours between administration of each dose, in particular wherein each dose is administered with food;
A certain embodiment of the present invention relates to the compound of formula (I), or a pharmaceutically acceptable salt thereof, for use in the treatment of BRAF-mutant metastatic melanoma with brain metastases, wherein the compound of formula (I) is administered orally with or without food;
A certain embodiment of the present invention relates to the compound of formula (I), or a pharmaceutically acceptable salt thereof, for use in the treatment of BRAF-mutant metastatic melanoma with brain metastases, wherein the compound of formula (I) is administered orally with food, in particular after a high fat meal;
A certain embodiment of the present invention relates to the compound of formula (I), or a pharmaceutically acceptable salt thereof, for use in the treatment of BRAF-mutant metastatic melanoma with brain metastases, wherein the compound of formula (I) is administered orally without food, in particular following a 10 hour fast;
A certain embodiment of the present invention relates to the compound of formula (I), or a pharmaceutically acceptable salt thereof, for use in the treatment of BRAF-mutant metastatic melanoma with brain metastases, wherein about 1600 mg/day to about 4000 mg/day of the Compound of formula (I) are administered orally, in particular 2000 mg/day to 4000 mg/day, more particular 3000 mg/day to 4000 mg/day;
A certain embodiment of the present invention relates to the compound of formula (I), or a pharmaceutically acceptable salt thereof, for use in the treatment of BRAF-mutant metastatic or locally advanced solid tumours, wherein about 1600 mg/day to about 4000 mg/day of the Compound of formula (I) are administered orally, in particular 2000 mg/day to 4000 mg/day, more particular 3000 mg/day to 4000 mg/day;
A certain embodiment of the present invention relates to the compound of formula (I), or a pharmaceutically acceptable salt thereof, for use in the treatment of non-small cell lung cancer, wherein 200 mg/day to 2000 mg/day of the Compound of formula (I) are administered orally, in particular 600 mg/day to 2000 mg/day, more particular 1000 mg/day to 2000 mg/day;
A certain embodiment of the present invention relates to the compound of formula (I), or a pharmaceutically acceptable salt thereof, for use in the treatment of non-small cell lung cancer, wherein about 1600 mg/day to about 4000 mg/day of the Compound of formula (I) are administered orally, in particular about 2000 mg/day to 4000 mg/day, more particular about 3000 mg/day to about 4000 mg/day;
A certain embodiment of the present invention relates to the compound of formula (I), or a pharmaceutically acceptable salt thereof, for use in the treatment of non-small cell lung cancer, wherein 1200 mg/day to 2000 mg/day of the Compound of formula (I) are administered orally, in particular 1400 mg/day to 2000 mg/day, more particular 1600 mg/day to 2000 mg/day;
A certain embodiment of the present invention relates to the compound of formula (I), or a pharmaceutically acceptable salt thereof, for use in the treatment of non-small cell lung cancer, wherein 800 mg/day to 1600 mg/day of the Compound of formula (I) are administered orally, in particular 800 mg/day to 1400 mg/day, more particular 1000 mg/day to 1400 mg/day;
A certain embodiment of the present invention relates to the compound of formula (I), or a pharmaceutically acceptable salt thereof, for use in the treatment of BRAF-mutant metastatic melanoma with brain metastases, wherein 50 mg/day to 800 mg/day of the Compound of formula (I) are administered orally during every day of a 28-day cycle, while cobimetinib is administered on days 1 to 21 of the 28-day cycle;
A certain embodiment of the present invention relates to the compound of formula (I), or a pharmaceutically acceptable salt thereof, for use in the treatment of BRAF-mutant metastatic melanoma with brain metastases, wherein 800 mg/day to 2000 mg/day of the Compound of formula (I) are administered orally during every day of a 28-day cycle, while cobimetinib is administered on days 1 to 21 of the 28-day cycle;
A certain embodiment of the present invention relates to the compound of formula (I), or a pharmaceutically acceptable salt thereof, for use in the treatment of BRAF-mutant metastatic melanoma with brain metastases, wherein about 2000 mg/day to about 4000 mg/day of the Compound of formula (I) are administered orally during every day of a 28-day cycle, while cobimetinib is administered on days 1 to 21 of the 28-day cycle;
A certain embodiment of the present invention relates to the compound of formula (I), or a pharmaceutically acceptable salt thereof, for use in the treatment of BRAF-mutant metastatic melanoma with brain metastases, wherein about 3000 mg/day to about 4000 mg/day, in particular about 3200 mg/day of the Compound of formula (I) are administered orally during every day of a 28-day cycle, while cobimetinib is administered on days 1 to 21 of the 28-day cycle;
A certain embodiment of the present invention relates to the compound of formula (I), or a pharmaceutically acceptable salt thereof, for use in the treatment of BRAF-mutant metastatic melanoma with brain metastases, wherein 1200 mg/day to 2000 mg/day, in particular 1600 mg/day, of the Compound of formula (I) are administered orally during every day of a 28-day cycle, while cobimetinib is administered on days 1 to 21 of the 28-day cycle;
A certain embodiment of the present invention relates to the compound of formula (I), or a pharmaceutically acceptable salt thereof, for use in the treatment of BRAF-mutant metastatic melanoma with brain metastases, wherein 800 mg/day to 1200 mg/day of the Compound of formula (I) are administered orally during every day of a 28-day cycle, while cobimetinib is administered on days 1 to 21 of the 28-day cycle;
A certain embodiment of the present invention relates to the compound of formula (I), or a pharmaceutically acceptable salt thereof, for use in the treatment of BRAF-mutant metastatic melanoma with brain metastases, wherein 50 mg/day to 800 mg/day, in particular 200 mg/day to 600 mg/day, of the Compound of formula (I) are administered orally during every day of a 28-day cycle, while 60 mg/day of cobimetinib is administered on days 1 to 21 of the 28-day cycle;
A certain embodiment of the present invention relates to the compound of formula (I), a pharmaceutically acceptable salt thereof, or a pharmaceutical composition as described herein, for use as described herein, wherein administration is performed twice a day (BID);
A certain embodiment of the present invention relates to the compound of formula (I), a pharmaceutically acceptable salt thereof, or a pharmaceutical composition comprising the compound of formula (I), for use as described herein, wherein 800 mg of the compound of formula (I) is adminstered twice a day (BID);
A certain embodiment of the present invention relates to the compound of formula (I), a pharmaceutically acceptable salt thereof, or a pharmaceutical composition comprising the compound of formula (I), for use as described herein, wherein 1200 mg of the compound of formula (I) is adminstered twice a day (BID);
A certain embodiment of the present invention relates to the compound of formula (I), a pharmaceutically acceptable salt thereof, or a pharmaceutical composition comprising the compound of formula (I), for use as described herein, wherein 1600 mg of the compound of formula (I) is adminstered twice a day (BID);
A certain embodiment of the present invention relates to the compound of formula (I), a pharmaceutically acceptable salt thereof, or a pharmaceutical composition comprising the compound of formula (I), for use as described herein, wherein 2000 mg of the compound of formula (I) is adminstered twice a day (BID);
A certain embodiment of the present invention relates to the compound of formula (I), a pharmaceutically acceptable salt thereof, or a pharmaceutical composition comprising the compound of formula (I), for use as described herein, wherein 800 mg of the compound of formula (I) is adminstered three-times a day (TID);
A certain embodiment of the present invention relates to the compound of formula (I), a pharmaceutically acceptable salt thereof, or a pharmaceutical composition comprising the compound of formula (I), for use as described herein, wherein 800 mg of the compound of formula (I) is adminstered three-times a day (TID) in intervals of 6 hours between the first and the second daily dose, respectively the second and third daily dose;
A certain embodiment of the present invention relates to the compound of formula (I), a pharmaceutically acceptable salt thereof, or a pharmaceutical composition comprising the compound of formula (I), for use as described herein, wherein 1200 mg of the compound of formula (I) is adminstered three-times a day (TID);
A certain embodiment of the present invention relates to the compound of formula (I), a pharmaceutically acceptable salt thereof, or a pharmaceutical composition comprising the compound of formula (I), for use as described herein, wherein 1200 mg of the compound of formula (I) is adminstered three-times a day (TID) in intervals of 6 hours between the first and the second daily dose, respectively the second and third daily dose;
A certain embodiment of the present invention relates to the compound of formula (I), a pharmaceutically acceptable salt thereof, or a pharmaceutical composition comprising the compound of formula (I), for use as described herein, wherein 1600 mg of the compound of formula (I) is adminstered three-times a day (TID);
A certain embodiment of the present invention relates to the compound of formula (I), a pharmaceutically acceptable salt thereof, or a pharmaceutical composition as described herein, for use as described herein, wherein administration is performed three-times a day (TID);
A certain embodiment of the present invention relates to the compound of formula (I), a pharmaceutically acceptable salt thereof, or a pharmaceutical composition as described herein, for use as described herein, wherein administration is performed three-times a day (TID) in intervals of 6 hours between the first and the second daily dose, respectively the second and third daily dose;
A certain embodiment of the present invention relates to the compound of formula (I), or a pharmaceutically acceptable salt thereof, for use in the treatment of BRAF-mutant cutaneous melanoma with asymptomatic brain metastases; and
A certain embodiment of the present invention relates to the compound of formula (I), or a pharmaceutically acceptable salt thereof, alone or in combination with cobimetinib, for use in the treatment of BRAF-mutant cutaneous melanoma with radiologically confirmed asymptomatic brain metastases.

### SPECIFIC NUMBERED EMBODIMENTS

1. A compound of formula (I)

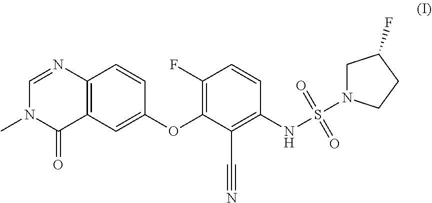 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in the treatment of melanoma with brain metastases.
2. The compound of formula (I) or a pharmaceutically acceptable salt thereof, for use according to embodiment 1, wherein the brain metastases are asymptomatic.
3. The compound of formula (I) or a pharmaceutically acceptable salt thereof, for use according to embodiment 1 or 2, wherein the melanoma is cutaneous melanoma.
4. The compound of formula (I) or a pharmaceutically acceptable salt thereof, for use according to any one of embodiments 1 to 3, wherein the melanoma has previously been treated with a BRAF inhibitor.
5. The compound of formula (I) or a pharmaceutically acceptable salt thereof, for use according to embodiment 4, wherein the BRAF inhibitor is selected from vemurafenib, dabrafenib and encorafenib.
6. The compound of formula (I) or a pharmaceutically acceptable salt thereof, for use according to any one of embodiments 1 to 3, wherein the melanoma has not previously been treated with a BRAF inhibitor.
7. The compound of formula (I) or a pharmaceutically acceptable salt thereof, for use according to any one of embodiments 1 to 6, wherein the compound of formula (I) is combined with a MEK inhibitor.
8. The compound of formula (I) or a pharmaceutically acceptable salt thereof, for use according to any one of embodiments 1 to 7, wherein the compound of formula (I) is combined with cobimetinib.
9. A method for the treatment or prophylaxis of melanoma with brain metastases, which method comprises administering an effective amount of the compound of formula (I)

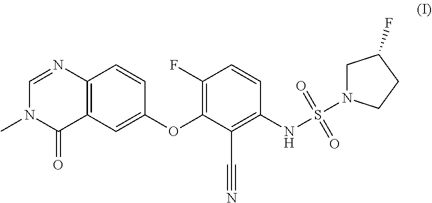 [SMILES: Cn1cnc2ccc(Oc3c(F)ccc(NS(=O)(=O)N4CC[C@@H](F)C4)c3C#N)cc2c1=O]

or a pharmaceutically acceptable salt thereof to a patient in need thereof.
10. The method according to embodiment 9, wherein the brain metastases are asymptomatic.
11. The method according to embodiment 9 or 10, wherein the melanoma is cutaneous melanoma.
12. The method according to any one of embodiments 9 to 11, wherein the melanoma has previously been treated with a BRAF inhibitor.
13. The method according to embodiment 12, wherein the BRAF inhibitor is selected from vemurafenib, dabrafenib and encorafenib.
14. The method according to any one of embodiments 9 to 11, wherein the melanoma has not previously been treated with a BRAF inhibitor.
15. The method according to any one of embodiments 9 to 14, wherein the compound of formula (I) is combined with a MEK inhibitor.
16. The method according to any one of embodiments 9 to 14, wherein the compound of formula (I) is combined with cobimetinib.
17. The use of a compound of formula (I)

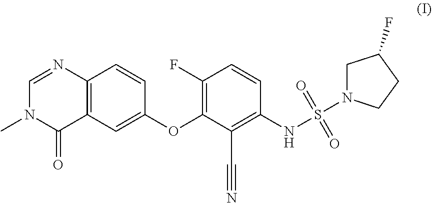 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for the treatment of melanoma with brain metastases.
18. The use according to embodiment 17, wherein the brain metastases are asymptomatic.
19. The use according to embodiment 17 or 18, wherein the melanoma is cutaneous.
20. The use according to any one of embodiments 17 to 19, wherein the melanoma has previously been treated with a BRAF inhibitor.
21. The use according to embodiment 20, wherein the BRAF inhibitor is selected from vemurafenib, dabrafenib and encorafenib.
22. The use according to any one of embodiments 17 to 19, wherein the melanoma has not previously been treated with a BRAF inhibitor.
23. The use according to any one of embodiments 17 to 22, wherein the compound of formula (I) is combined with a MEK inhibitor.
24. The use according to any one of embodiments 17 to 23, wherein the compound of formula (I) is combined with cobimetinib.
25. A pharmaceutical composition comprising a compound of formula (I)

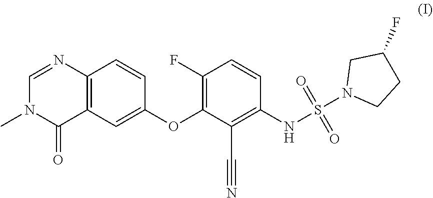 [SMILES: Cn1cnc2ccc(Oc3c(F)ccc(NS(=O)(=O)N4CC[C@@H](F)C4)c3C#N)cc2c1=O]

or a pharmaceutically acceptable salt thereof and at least one therapeutically inert carrier, for use in the treatment of melanoma with brain metastases.
26. The pharmaceutical composition for use according to embodiment 25, wherein the brain metastases are asymptomatic.
27. The pharmaceutical composition for use according to embodiment 25 or 26, wherein the melanoma is cutaneous melanoma.
28. The pharmaceutical composition for use according to any one of embodiments 25 to 27, wherein the melanoma has previously been treated with a BRAF inhibitor.
29. The pharmaceutical composition for use according to any one of embodiments 25 to 28, wherein the BRAF inhibitor is selected from vemurafenib, dabrafenib and encorafenib.
30. The pharmaceutical composition for use according to any one of embodiments 25 to 27, wherein the melanoma has not previously been treated with a BRAF inhibitor.
31. The pharmaceutical composition for use according to any one of embodiments 25 to 30, wherein the compound of formula (I) or a pharmaceutically acceptable salt thereof, is combined with a MEK inhibitor.
32. The pharmaceutical composition for use according to any one of embodiments 25 to 31, wherein the compound of formula (I), or a pharmaceutically acceptable salt thereof, is combined with cobimetinib.
33. A pharmaceutical composition comprising a compound of formula (I)

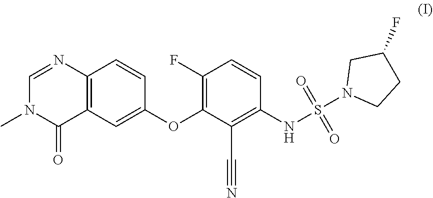 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, wherein the pharmaceutical composition comprises one or more fillers, a glidant, a disintegrant, and a lubricant, preferably the pharmaceutical composition comprises one or more fillers, a glidant, a disintegrant, a pH-modifier, a lubricant, and a wetting agent
34. The pharmaceutical composition of embodiment 33, wherein the pharmaceutical composition comprises a film coat.
35. The pharmaceutical composition of embodiment 34, wherein the film coat comprises one or more components selected from polyvinyl alcohol, macrogol 3350, polyethylene glycol 4000, Talc, titanium dioxide, iron oxide yellow and iron oxide red.
36. The pharmaceutical composition of any one of embodiments 33 to 35, wherein the fillers are selected from microcrystalline cellulose, lactose monohydrate, anhydrous lactose, mannitol, isomalt and dibasic dicalcium phosphate.
37. The pharmaceutical composition of any one of embodiments 33 to 36, wherein the fillers are selected from microcrystalline cellulose and lactose monohydrate.
38. The pharmaceutical composition of any one of embodiments 33 to 37, wherein the fillers are microcrystalline cellulose and lactose monohydrate in a ratio of about 2:1 (microcrystalline cellulose:lactose monohydrate) weight percent.
39. The pharmaceutical composition of any one of embodiments 33 to 38, wherein the weight percentage of the filler or the combined weight percentage of the fillers is between about 30 weight percent to about 90 weight percent of the pharmaceutical composition.
40. The pharmaceutical composition of any one of embodiments 33 to 39, wherein the wetting agent is selected from Sodium Lauryl Sulfate, PVP/PVA copolymer and hypromellose in a weight percentage range of about 0.1 weight percent to about 5.0 weight percent of the pharmaceutical composition.
41. The pharmaceutical composition of any one of embodiments 33 to 40, wherein the wetting agent is Sodium Lauryl Sulfate in a weight percentage of about 0.1 weight percent of the pharmaceutical composition.
42. The pharmaceutical composition of any one of embodiments 33 to 40, wherein the weight percentage of the glidant is about 0.5 weight percent of the pharmaceutical composition.
43. The pharmaceutical composition of any one of embodiments 33 to 42, wherein the glidant is colloidal silicon dioxide.
44. The pharmaceutical composition of any one of embodiments 33 to 43, wherein the pH-modifier is an alkaline pH-modifier.
45. The pharmaceutical composition of any one of embodiments 33 to 44, wherein the pH-modifier is selected from magnesium oxide, calcium carbonate, calcium hydrogen carbonate, lysine and tromethamine.
46. The pharmaceutical composition of any one of embodiments 33 to 45, wherein the pH-modifier is magnesium oxide.
47. The pharmaceutical composition of any one of embodiments 33 to 46, wherein the weight percent ratio of pH-modifier to the compound of formula (I) is between about 3:1 and about 1:5.
48. The pharmaceutical composition of any one of embodiments 33 to 47, wherein the weight percent ratio of pH-modifier to the compound of formula (I) is about 1:2.
49. The pharmaceutical composition of any one of embodiments 33 to 48, wherein the disintegrant is selected from crospovidone, sodium starch glycolate, L-HPC and Sodium Croscarmellose.
50. The pharmaceutical composition of any one of embodiments 33 to 49, wherein the disintegrant is Sodium Croscarmellose.
51. The pharmaceutical composition of any one of embodiments 33 to 50, wherein the weight percentage of the disintegrant is about 2 weight percent to 5 weight percent of the pharmaceutical composition.
52. The pharmaceutical composition of any one of embodiments 33 to 51, wherein the weight percentage of the disintegrant is about 3 weight percent of the pharmaceutical composition.
53. The pharmaceutical composition of any one of embodiments 33 to 52, wherein part of the disintegrant is added prior to dry granulation and part of the disintegrant is added after dry granulation.
54. The pharmaceutical composition of any one of embodiments 33 to 53, wherein the disintegrant contains at least one precipitation inhibitor selected from HPMC, HPMC-AS, polyvinyl caprolactam-polyvinyl acetate-polyethylene glycol graft copolymer (PCL-PVAc-PEG), PVP/PVA copolymer in weight percentage range of 5 weight percent to 10 weight percent of the disintegrant.
55. The pharmaceutical composition of any one of embodiments 33 to 54, wherein the lubricant is selected from magnesium stearate, sodium stearyl fumarate and macrogol 6000.
56. The pharmaceutical composition of any one of embodiments 33 to 55, wherein the lubricant is magnesium stearate.
57. The pharmaceutical composition of any one of embodiments 33 to 56, wherein the weight percentage of the lubricant is about 0.5 weight percent to about 3.0 weight percent of the pharmaceutical composition.
58. The pharmaceutical composition of any one of embodiments 33 to 57, wherein the weight percentage of the lubricant is about 0.5 weight percent to about 1.0 weight percent of the pharmaceutical composition.
59. The pharmaceutical composition of any one of embodiments 34 to 58, wherein the film coat contains HPMC.
60. The pharmaceutical composition of any one of embodiments 34 to 59, wherein the weight percentage of the film coat is between about 2.0 weight percent to about 5.0 weight percent of the pharmaceutical composition.
61. The pharmaceutical composition of any one of embodiments 34 to 60, wherein the weight percentage of the film coat is about 3.0 weight percent of the pharmaceutical composition.
62. The pharmaceutical composition of any one of embodiments 25 to 61, wherein the pharmaceutical composition comprises Sodium Lauryl Sulfate, Microcrystalline Cellulose, Lactose monohydrate, Magnesium Oxide, Colloidal silicon dioxide, Sodium Croscarmellose and Magnesium stearate.
63. The pharmaceutical composition of any one of embodiments 25 to 62, wherein the pharmaceutical composition comprises an enteric coating.
64. The pharmaceutical composition of any one of embodiments 25 to 62, wherein the pharmaceutical composition comprises an enteric coating comprising at least one, two or three components selected from methyl acrylate-methacrylic acid copolymers, cellulose acetate phthalate, cellulose acetate succinate, hydroxypropyl methyl cellulose phthalate, hydroxypropyl methyl cellulose acetate succinate (hypromellose acetate succinate), polyvinyl acetate phthalate, (PVAP), methyl methacrylate-methylacrylic acid copolymers, shellac, cellulose acetate trimellitate, sodium alginate and zein.
65. The compound for use, pharmaceutical composition, method or use according to any one of embodiments 7, 15, 23 or 31, wherein the MEK inhibitor is selected from binimetinib, trametinib and cobimetinib.
66. The compound for use, pharmaceutical composition, method or use according to any one of embodiments 4-5, 12-13, 20-21 or 28-29, wherein relapse occurred under previous treatment with a BRAF inhibitor.
67. The compound for use, pharmaceutical composition, method or use according to any one of embodiments 1-32 or 65-66, wherein the melanoma is associated with BRAF^{V600} mutations.
68. A compound of formula (I)

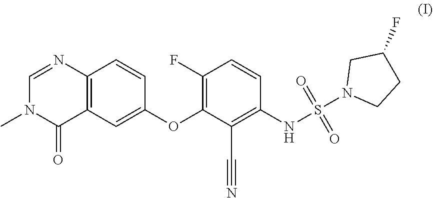 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, for use in locally advanced solid tumour and/or cancer metastases.
69. The compound of formula (I) or a pharmaceutically acceptable salt thereof, for use according to embodiment 68, wherein the metastatic cancer is characterized by having at least one metastatic site in the brain.
70. The compound of formula (I) or a pharmaceutically acceptable salt thereof, for use according to embodiment 68 or 69, wherein the cancer metastases are asymptomatic.
71. The compound of formula (I) or a pharmaceutically acceptable salt thereof, for use according to any one of embodiments 68 to 70, wherein the locally advanced solid tumour is selected from melanoma, non-small cell lung cancer (NSCLC), thyroid cancer, colorectal cancer (CRC), in particular non-small cell lung cancer (NSCLC).
72. The compound of formula (I) or a pharmaceutically acceptable salt thereof, for use according to any one of embodiments 68 to 71, wherein the metastatic or locally advanced solid tumors has previously been treated with a BRAF inhibitor.
73. The compound of formula (I) or a pharmaceutically acceptable salt thereof, for use according to embodiment 72, wherein the BRAF inhibitor is selected from vemurafenib, dabrafenib and encorafenib.
74. The compound of formula (I) or a pharmaceutically acceptable salt thereof, for use according to any one of embodiments 68 to 71, wherein the solid tumour has not previously been treated with a BRAF inhibitor.
75. The compound of formula (I) or a pharmaceutically acceptable salt thereof, for use according to any one of embodiments 68 to 74, wherein the compound of formula (I) is combined with a MEK inhibitor.
76. The compound of formula (I) or a pharmaceutically acceptable salt thereof, for use according to any one of embodiments 68 to 75, wherein the compound of formula (I) is combined with cobimetinib.
77. A pharmaceutical composition having an approximate weight to weight ratio (relative to the mass of the whole composition) according to any one of examples A, B or C, wherein the weight to weight ratio can deviate from the examples with not more than 20%, preferably not more than 10%.
78. A pharmaceutical composition according to any one of examples A, B or C.

1-15. (canceled)

16. A pharmaceutical composition comprising a compound of formula (I): 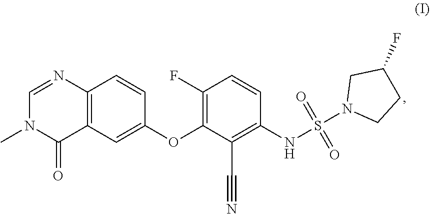 [SMILES: Cn1cnc2ccc(Oc3c(F)ccc(NS(=O)(=O)N4CC[C@@H](F)C4)c3C#N)cc2c1=O]
or a pharmaceutically acceptable salt thereof,
wherein the pharmaceutical composition comprises one or more fillers, a glidant, a disintegrant, a pH-modifier, a lubricant, and a wetting agent.

17. The pharmaceutical composition of claim 16, wherein the pH-modifier is an alkaline pH-modifier.

18. The pharmaceutical composition of claim 17, wherein the pH-modifier is selected from magnesium oxide, calcium carbonate, calcium hydrogen carbonate, lysine, and tromethamine.

19. The pharmaceutical composition of claim 18, wherein the pH-modifier is magnesium oxide.

20. The pharmaceutical composition of claim 16, wherein the pharmaceutical composition comprises Sodium Lauryl Sulfate, Microcrystalline Cellulose, Lactose monohydrate, Magnesium Oxide, Colloidal silicon dioxide, Sodium Croscarmellose, and Magnesium stearate.

21. The pharmaceutical composition of claim 16, wherein the weight percent ratio of pH-modifier to the compound of formula (I) is between about 3:1 and about 1:5.

22. The pharmaceutical composition of claim 16, wherein the weight percent ratio of pH-modifier to the compound of formula (I) is between about 1:2.

23. The pharmaceutical composition of claim 16, wherein the pharmaceutical composition comprises a film coat.

24. The pharmaceutical composition of claim 16, wherein the pharmaceutical composition comprises an enteric coating.

25. The pharmaceutical composition of claim 24, wherein the pharmaceutical composition comprises an enteric coating comprising at least one, two, or three components selected from methyl acrylate-methacrylic acid copolymers, cellulose acetate phthalate, cellulose acetate succinate, hydroxypropyl methyl cellulose phthalate, hydroxypropyl methyl cellulose acetate succinate (hypromellose acetate succinate), polyvinyl acetate phthalate, (PVAP), methyl methacrylate-methylacrylic acid copolymers, shellac, cellulose acetate trimellitate, sodium alginate, and zein.

26. A method of threating a BRAF-mutant metastatic melanoma with brain metastases, the method comprising administering to a patient in need thereof an effective amount of a compound of formula (I): 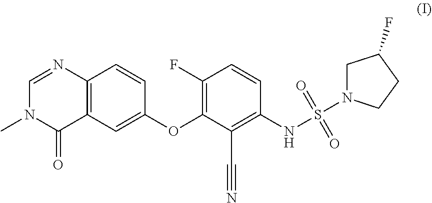 [SMILES: Cn1cnc2ccc(Oc3c(F)ccc(NS(=O)(=O)N4CC[C@@H](F)C4)c3C#N)cc2c1=O]
or a pharmaceutically acceptable salt thereof,
in combination with an effective amount of cobimetinib, or a pharmaceutically acceptable salt thereof,
wherein about 3000 mg/day to about 4000 mg/day of the compound of formula (I), or a pharmaceutically acceptable salt thereof, are administered orally during every day of a 28-day cycle, while cobimetinib, or a pharmaceutically acceptable salt thereof, is administered on days 1 to 21 of the 28-day cycle.

27. The method of claim 26, the compound of formula (I), or a pharmaceutically acceptable salt thereof, is administered in an effective amount of about 3200 mg/day.

28. A method of treating a locally advanced solid tumor and/or its cancer metastases, the method comprising administering to a patient in need thereof an effective amount of a compound of formula (I): 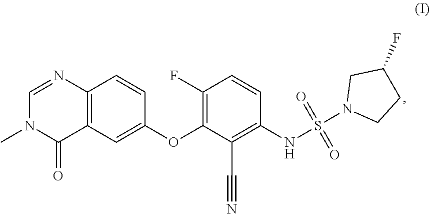 [SMILES: Cn1cnc2ccc(Oc3c(F)ccc(NS(=O)(=O)N4CC[C@@H](F)C4)c3C#N)cc2c1=O]
a pharmaceutically acceptable salt thereof,
wherein the administering is performed three-times a day (TID).

29. The method of claim 28, comprising administering the effective amount of about 1200 mg of the compound of formula (I), or a pharmaceutically acceptable salt thereof.

30. The method of claim 29, where the administering is performed in intervals of about 6 hours between the first and the second daily dose, respectively the second and third daily dose.

31. A method of treating a BRAF-mutant metastatic or locally advanced solid tumor, the method comprising administering to a patient in need thereof an effective amount of a compound of formula (I): 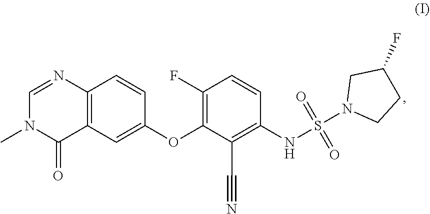 [SMILES: Cn1cnc2ccc(Oc3c(F)ccc(NS(=O)(=O)N4CC[C@@H](F)C4)c3C#N)cc2c1=O]
or a pharmaceutically acceptable salt thereof,
wherein the compound of formula (I), or a pharmaceutically acceptable salt thereof, is administered orally with food.

32. The method of claim 31, wherein the compound of formula (I), or a pharmaceutically acceptable salt thereof, is administered after a high-fat meal.
